(19) United States
(12) Patent Application Publication
(10) Pub. No.: US 2005/0165323 A1
(43) Pub. Date: Jul. 28, 2005

(54) Physiological signal monitoring apparatus and method

(75) Inventors: Anthony R. Montgomery, Verona, WI (US); Jason W. Bold, Verona, WI (US); Douglas Carpiaux, Milwaukee, WI (US); Ming Chang, Terrey Hills (AU); Douglas M. Code, Madison, WI (US); Ernest C. Jacobs, Blue Mounds, WI (US); Scott H. Micoley, Cedarburg, WI (US); Jeffrey P. Milsap, Cambridge, WI (US); Peter G. Montgomery, Hornsby Heights (AU); Robert L. Palmer, Cremorne (AU); Daniel C. Strelow, Barneveld, WI (US); Jeffrey Wierschke, Sheboygan Falls, WI (US); Elaine Jamieson, Cremorne (AU)

Correspondence Address: Michael Best & Friedrich LLP, 100 East Wisconsin Avenue, Milwaukee, WI, 53202-4108, US

(73) Assignee: LaMont, LLC.

(21) Appl. No.: 10/848,968

(22) Filed: May 19, 2004

Related U.S. Application Data
(63) Continuation of application No. 09/680,882, filed on Oct. 6, 2000, now abandoned.
(60) Provisional application No. 60/158,200, filed on Oct. 7, 1999.

(51) Int. Cl. A61B 5/04

(52) U.S. Cl. 600/544; 128/903

(57) ABSTRACT

Preferred embodiments of the invention employ a portable and wearable EEG monitoring device having a patient-worn amplifier releasably coupled to a host computer for transmitting EEG signals. When patient disconnection from the host computer is desired, a portable operations device (POD) can be connected to the amplifier. Preferably upon detecting disconnection, a controller causes new EEG signals to be routed to a removable memory or transmitter peripheral card, enabling seamless data acquisition. Upon detecting reconnection between the amplifier and the host computer, the controller causes new EEG signals to be routed to the host computer. The controller also preferably transmits EEG signals stored on the peripheral memory card (if used) to the host computer. Preferred embodiments include a handheld display apparatus for viewing EEG signals and electrode information. Also, preferred embodiments reduce patient tethers by connecting multiple amplifiers in a daisy-chain format (most preferably on a PAN bus).

### RELATED APPLICATIONS

Priority is claimed to U.S. Patent Application Ser. No. 60/158,200.

### FIELD OF THE INVENTION

This invention relates generally to physiological monitoring and control, and more particularly to apparatuses and methods for monitoring and controlling physiological processes of a patient.

### BACKGROUND OF THE INVENTION

Electroencephalograms (EEGs) record the oscillating electrical activity within the brain, i.e. the electrical potential fluctuations within the brain. The brain is basically a large conductive medium containing an array of active neuronal elements. EEGs record the total resultant field potential of this array of active neuronal elements. Large numbers of neuronal elements must be synchronously active to give rise to potentials recorded from the brain surface.

Conventionally, the electrical activity of the brain is recorded with one of three types of electrodes, namely scalp, cortical, and depth electrodes. Scalp electrodes are attached to the skin of the scalp, either between hair follicles or on a shaved scalp. Cortical electrodes are placed on the exposed surface of the brain, referred to as the cortex. Depth electrodes are thin insulated electrodes that are advanced directly into the neural tissue of the brain.

Different noise and interference problems exist for each type of electrode. Of course, the further the electrode is from the source of the signal and the more matter between the electrode and the source of the signal, the more noise and interference in the detected signal. Accordingly, depth electrode signals have the least noise and interference, whereas scalp electrode signals have the most noise and interference. For example, the intensity of the brain waves on the surface of the brain or cortex may be as large as 10 millivolts, whereas the brain waves recorded from the scalp have an amplitude of 100 microvolts. Noise and interference problems exist with conventional EEG monitoring using scalp electrodes due to the level of amplification and filtration necessary to detect a clinically significant signal.

The clinically significant frequencies of brain waves typically range from 0.5 Hz to more than 100 Hz, and brain wave characteristics depend upon the degree of activity of the cerebral cortex. The characteristics of brain waves change between wakefulness and sleep. Much of the time brain waves are irregular and no pattern can be observed. However, patterns do occur in special abnormalities, such as epilepsy, sleep disorders, or nystagmus. Epilepsy is the uncontrolled excessive activity by a part or all of the central nervous system. Epileptic seizures occur when the basal level of excitability of the central nervous system rises above a certain critical threshold. Some examples of sleep disorders include obstructive sleep apnea, REM sleep behavior disorder, and restless legs syndrome. Nystagmus is the rhythmic oscillation of the eyeballs, either pendular or jerky.

Patients with disorders, such as epilepsy, sleep disorders, and nystagmus, often need to be monitored continuously over long periods of time in order to study, diagnose, and treat these disorders. The need to monitor the brain waves of patients for long periods of time creates many problems relating to the ergonomic design and portability of EEG monitoring devices. Additionally, patients being monitored over long periods of time cannot normally be constantly connected to physiological monitors coupled to central analysis and storage stations. For example, patients must often be disconnected from such monitors when being transported (e.g., to a bathroom, to another area of a facility, or between facilities), bathed, etc. When a patient is not coupled to a monitoring device, problems occur with data loss. These problems are exacerbated because the very brain activity desired to be monitored often sometimes when the patient is being moved or otherwise disturbed—the very times when many conventional monitoring devices are disconnected by necessity or convenience. Even if the EEG data is somehow stored while the patient is not coupled to the monitoring device, problems occur with synchronizing the stored data with the previously recorded data and with the new incoming data.

Development of portable devices intended to be worn more continuously than conventional EEG monitoring equipment has been hampered by the demands placed upon such systems. For example, the power requirements for amplifying and processing potentially more than a hundred signals from electrodes on the patient are demanding. In addition, a reliable manner in which a portable system can be connected and disconnected to a central station during patient monitoring and without loss of data has not been developed prior to the present invention.

Another common problem with conventional EEG monitoring systems is related to the complexity, size, connectability, and weight of such systems. Typically, conventional systems employ multiple devices connected together via cables or other wiring. The various devices include jackboxes, amplifiers, and the like. These separate devices are difficult to manage and can easily become disorganized, occupy valuable space around the patient, decrease the patient's ability to move freely, and increase patient discomfort. Because conventional amplifiers used in these systems have limited electrode capacities, multiple amplifiers each having at least one cable connection to a central station are commonly used. For obvious reasons, multiple cables generate further problems such as those just described. Due at least in part to their inherent complexity, conventional EEG monitoring systems are also poorly suited for mobile use. Such systems are not intended to be portable, and are typically designed for use within a limited range in a facility. Accordingly, their usefulness is usually limited by their inability to operate outside of the facility (and often even outside of a range while still within the facility).

Conventional patient monitoring systems generally have a fixed number of amplifiers within a given system. Additional amplifiers, whether made by the same manufacturer or by a different manufacturer, usually cannot easily be added to the monitoring system while recording data. Also, additional amplifiers cannot by added to conventional EEG monitoring systems while monitoring is in process without risking data loss or corruption.

Brain waves are generally classified into four groups: alpha, beta, theta, and delta. Alpha waves are rhythmic with a frequency range of 8 to 13 Hz. The amplitude of alpha waves is about 20 to 200 microvolts. Alpha waves are detected when patients are awake, but in a quiet resting state. Alpha waves disappear when a patient is asleep. Beta waves have a frequency range of 14 to 30 Hz, and may be as high as 50 Hz during intense mental activity. There are two types of beta waves, one of which is elicited by mental activity, the other of which is inhibited by mental activity. Theta waves have a frequency range of 4 to 7 Hz. Theta waves are detected mainly in children, but also during emotional stress in adults. Delta waves include all the brain waves below 3.5 Hz, and are sometimes only detected every two or three seconds. As can be seen by the various frequency ranges of the four types of brain waves, the need to monitor brain waves in several different frequency ranges presents significant design problems. Additionally, problems occur in designing an amplifier for waves of such low amplitude.

Another problem with conventional EEG monitoring systems is the ability of a user to quickly and easily view the EEG signals, view impedance measurements of the electrodes to determine the quality of electrode connections to the patient, change the threshold for electrode impedances, calibrate the system to verify proper operation, and view other patient physiological data. Each of these activities must typically be performed not only while the patient is tethered to a central station, but also while a station monitor is in view. This presents problems for technicians and staff when the central station capable of displaying monitoring system information is not located near the patient being monitored (or at least in easy view from the patient's location). Users of conventional systems must either move the patient near a monitor capable of displaying such monitoring system information, install redundant monitors in multiple locations, or must leave the patient to view this information. These options are undesirable and represent yet another deficiency in conventional EEG monitoring systems.

Although the problems and limitations described above are with reference to conventional EEG monitoring systems (discussed herein by way of example only), these problems and limitations apply to many other types of patient monitoring, including without limitation sleep monitoring, heart monitoring, maternal/fetal monitoring, respiratory monitoring, ambulatory monitoring and the like.

In light of the problems and limitations of the prior art described above, a need exists for an apparatus and method for monitoring physiological signals in which the monitoring apparatus is portable, compact, comfortable to wear, and has reduced cabling between the patient and a central station, connection and disconnection from a central station is possible even during patient monitoring without the loss or corruption of data, a user can quickly and easily view physiological signals and information and change system operation even if away from the central station or a central station monitor, the monitoring apparatus is modular in that multiple amplifiers can be connected even during patient monitoring without data corruption or loss, physiological signal data can be acquired even if the apparatus is disconnected from the central station for extended periods of time and can be repatriated with earlier or later-acquired data on the central station without data loss or corruption even at the same time data acquisition is in process, data from multiple amplifiers is properly synchronized and processed, and physiological signals in different frequency ranges can be monitored. Each preferred embodiment of the present invention achieves one or more of these results.

### SUMMARY OF THE INVENTION

The present invention relates to the monitoring and control of physiological signals, preferably electroencephalographic (EEG) signals. In preferred embodiments of the present invention, an amplifier on the patient is releasably coupled to a stationary or mobile host computer for transmitting a patient's EEG signals received by the amplifier. The amplifier can be coupled or “tethered” to the host computer via a cable connected thereto or via wireless transmission of data. EEG signals are thereby transmitted to the host computer from the amplifier along the cable, while power is preferably supplied by the cable to the amplifier and the rest of the apparatus.

When patient disconnection from the host computer is desired, a portable operations device is connected to the amplifier. A battery connected to the portable operations device can supply power to the portable operations device, to the amplifier, and to the rest of the apparatus while disconnected from the host computer. Alternatively, the portable operations device is connected to and powered by a mains power supply, which generally provides 120VAC or 240VAC power from outlets in the walls of buildings. The portable operations device is connected to a mains power supply for desktop monitoring units located away from the actual host computer. The portable operations device preferably has a controller capable of controlling the apparatus when disconnected from the host computer. Preferably, the portable operations device can be connected to the amplifier without disturbing the cable connection thereto, and establishes electrical communication with the amplifier via a communications port or jack separate from the communications port or jack to which the cable is connected. Once the portable operations device is connected to the amplifier, the cable can be disconnected. Upon detecting disconnection of the cable between the amplifier and the host computer, the controller causes new EEG signals received by the amplifier to be routed to the portable operations device. New signals received from additional amplifiers added after the amplifier is disconnected from the host computer are also immediately routed to the portable operations device. The transfer of the stream of EEG signals from the amplifier to the portable operations device is seamless and thereby results in no loss or corruption of data.

The seamless transfer of the stream of EEG signals is accomplished by the controller of the portable operations device monitoring the peripheral area network in order to detect when the host computer is disconnected. Once the controller detects the disconnection, data is routed to the portable operations device for processing and storage. The data is stored within the portable operations device until the amplifier is reconnected to the host computer.

The portable operations device preferably has a housing and at least one bay in the housing coupled to the controller for removably receiving one or more peripheral cards. The peripheral cards can be memory cards, wireless transmitter cards, or network or modem cards, and can receive EEG signals from the amplifier when the amplifier is disconnected from the host computer. Where a memory card is used, EEG signals are transmitted via the bay to the peripheral memory card where they are stored. Where a wireless transmitter card is used, EEG signals are transmitted via the bay to the peripheral wireless transmitter card where they are preferably wirelessly transmitted to a wireless receiver on the host computer. Where a network or modem connection is used, EEG signals are transmitted via wires to a network connected to the host computer or to the host computer directly.

As used herein and in the appended claims, reference to an EEG signal or any other physiological signal (whether being transmitted, received, stored, processed, or otherwise) is intended to refer to the physiological signal and to data representative of the physiological signal in any form and in any location in the apparatus.

Upon detecting reconnection of the cable between the amplifier and the host computer, the controller causes new EEG signals received by the amplifier to be routed via a cable or via a network or modem to the host computer. At this or a later time, the controller also preferably transmits the EEG signals stored on the peripheral memory card (if used) to the host computer via transmitting these signals to the host computer via a cable or via a network or modem. Alternatively or in addition, the peripheral memory card can be removed from the portable operations device and can be connected to the host computer to repatriate the data thereon with earlier and/or later EEG data transmitted to the host computer.

In some preferred embodiments of the present invention, EEG signals are transmitted to the host computer by a wireless transmitter on the amplifier communicating with a wireless receiver on the host computer, rather than by cable. The EEG signals are transmitted directly to the host computer via the wireless transmitter. When a loss of wireless communication between the amplifier and the host computer is detected, the apparatus preferably operates in much the same manner as when the cable of the above-described embodiment is disconnected. During the loss of wireless communication, the EEG signals are stored to memory for a time period and then transmitted to the host computer once wireless communication is re-established. When re-establishment of this wireless communication is detected, the apparatus preferably operates in much the same manner as when the cable of the above-described embodiment is re-connected.

By employing a portable operations device having one or more peripheral cards, a constant stream of EEG data is acquired and synchronized with other patient data, such as digital video data, regardless of whether communication is lost with the host computer. Immediate or delayed repatriation of data stored to an on-board peripheral card memory or transmitted via a wireless transmitter peripheral card results in the uninterrupted synchronization of old and new EEG data and other patient data, such as digital video data, in contrast to the interrupted asynchronization of old and new EEG data of conventional systems.

If desired, one or more jacks or ports can be provided upon the amplifier and/or the portable operations device for connecting one or more extra physiological monitoring devices thereto. For example, an event marker pendent, an activation device or stimulator, and a pulse oximeter can be connected to the amplifier via dedicated jacks. A microphone jack, pneumatic ports, and high-level DC to 150 Hz inputs can be provided on the portable operations device or the amplifier for connection to a microphone, a breathing monitor, and other patient monitoring devices, respectively. Such physiological monitoring devices can even be built into the portable operations device (or amplifier), such as a microphone or a light sensor built into the portable operations device. Preferably, each of these physiological monitoring devices are connected to the controller of the portable operations device, to the amplifier (to transmit the additional signals to the host computer via the amplifier) and/or to the bays (to transmit the additional physiological signals to the peripheral cards when the amplifier is disconnected from the host computer).

In some highly preferred embodiments of the present invention, the amplifier has an expansion communications jack or port to which one or more additional amplifiers can be connected as desired, preferably even while acquiring data. The amplifiers not only acquire EEG data, but also other patient monitoring data, such as digital video data. Specifically, rather than connect an additional amplifier to the host computer by a dedicated cable, the cable can be connected to the expansion communications jack or port of an already-connected amplifier. The initial amplifier may or may not be on the portable operations device. Still other amplifiers can preferably be connected in this daisy-chain configuration, whereby an output of one amplifier is connected to the expansion communications jack or port of another amplifier. Therefore, unlike conventional monitoring systems, the addition of amplifiers to the apparatus of the present invention does not result in additional patient-to-host computer tethers.

To permit amplifiers to be added and removed from the apparatus without data loss or corruption even during patient monitoring, the cabling and connections between the amplifiers and the portable operations device is a peripheral area network bus specifically configured to the present invention. Accordingly, amplifiers can be “hot plugged” to or removed from an existing assembly as needed. In one highly preferred embodiment, additional amplifiers can be hot plugged while the patient is disconnected from the host computer 16 and the system is controlled by the portable operations device.

The hot plugging of additional amplifiers is accomplished by a signal from the controller being sent continuously over the peripheral area network bus to seek out additional amplifiers. As the data from each connected amplifier is collected by the controller, space is left for the possibility of additional amplifiers, with additional amplifier data being added into the data stream. For example, when two amplifiers are connected, two send signals are sent over the peripheral area network bus, along with two wait signals. The wait signals correspond to the possibility of two additional amplifiers being added to the first two amplifiers any time during monitoring.

In some preferred embodiments of the present invention, a handheld display apparatus is provided for viewing EEG signal information and, more preferably, for controlling apparatus operation via at least one user-manipulable control on the handheld display apparatus. The handheld display apparatus is preferably coupled to an amplifier of the EEG monitoring apparatus and has a display screen upon which EEG signal information can be viewed by a user. Preferably, the handheld display apparatus has an electrode test mode in which threshold impedance values can be selected by the user via user-manipulable controls and in which electrodes having measured impedances over their maximum threshold impedance values are indicated. The handheld display apparatus preferably also allows for user control of a calibration mode for calibrating electrodes and in which EEG traces corresponding to electrodes connected to the apparatus can be viewed, a pulse oximeter mode, and a waveform display mode. The information displayed on the handheld display unit (such as the electrode impedance values and the EEG traces) are preferably continuously updated. By employing a handheld display apparatus as just described, a user can view EEG signal information and/or can control apparatus operation (e.g., changing threshold impedance values of the electrodes) without needing to view the host computer monitor and in some cases without needing to input commands to the host computer. Apparatus setup is therefore faster and easier, and EEG signal and electrode information is more readily accessible than in conventional devices and systems.

In addition to reducing the number of cables connecting the patient to the host computer when multiple amplifiers are used, the present invention increases patient comfort by the manner in which the various elements of the apparatus are arranged and worn on the patient. Specifically, the amplifier and the battery are preferably mounted upon or integral with the portable operations device to define a single physically integral unit. This arrangement of devices in the apparatus is easier to wear and to results in an apparatus that can be more quickly set up on the patient. More preferably, the single physically integral unit also includes the jackbox to which the patient electrodes are connected, whereby the jackbox is mounted upon the amplifier. In alternative embodiments, the jackbox and/or amplifier can be worn on other areas of the patient and can be connected via cables of suitable length as desired.

For increased user comfort and wearability, the amplifier, portable operations device, and battery can be received within a holster worn on the patient. In a highly preferred embodiment, the holster is connected to a belt worn upon the patient. In this and other embodiments, a belt can be used to hold multiple amplifiers as well as the cable(s) connecting these amplifiers together in a manner as described above.

The foregoing description and the following detailed description is with reference to EEG monitoring and control. However, it should be noted that the present invention finds application in virtually any type of physiological monitoring. EEG monitoring is presented herein by way of example only and is not to be considered as a limiting factor of the present invention. The present invention is preferably used in monitoring electrical signals detected by electrodes or other sensors attached to the body of the patient in any conventional manner. By way of example only, the present invention could be configured to the monitoring and control of maternal/fetal signals, cardiac signals, sleep disorder signals, respiratory signals, and muscular signals. Additionally, video signals of a patient's appearance can be synchronized with any of the above-mentioned biopotential signals. In order to configure the present invention to other types of monitoring, different algorithms for each type of monitoring are downloaded into the controllers in order to implement the specific requirements for each type of monitoring.

Further objects and advantages of the present invention, together with the organization and manner of operation thereof, will become apparent from the following detailed description of the invention when taken in conjunction with the accompanying drawings, wherein like elements have like numerals throughout the drawings.

### BRIEF DESCRIPTION OF THE DRAWINGS

The present invention is further described with reference to the accompanying drawings, which show a preferred embodiment of the present invention. However, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a perspective view of a physiological signal monitoring apparatus according to a first preferred embodiment of the present invention, shown worn upon a patient;

FIG. 2 is a schematic view of the physiological signal monitoring apparatus shown in FIG. 1;

FIG. 3 is a perspective view of a physiological signal monitoring apparatus according to a second preferred embodiment of the present invention, shown worn upon a patient;

FIG. 4 is a schematic view of the physiological signal monitoring apparatus shown in FIG. 4;

FIGS. 5a and 5b are perspective views of the portable operations device, amplifier, and battery assembly shown in FIGS. 1-4, shown assembled into an integral unit;

FIG. 5c is a perspective view of the portable operations device, amplifier, and battery assembly shown in FIGS. 5a and 5b, shown installed within a holster;

FIG. 6a is an exploded perspective view of the jackbox and amplifier shown in FIGS. 1-4;

FIG. 6b is an exploded top view of the jackbox and amplifier shown in FIG. 6a;

FIG. 6c is an exploded side view of the jackbox and amplifier shown in FIGS. 6a and 6b;

FIG. 7 is a schematic view of the amplifier shown in FIGS. 1-4;

FIG. 8 is a schematic view of a host computer interface according to a first preferred embodiment of the present invention;

FIG. 9 is a schematic view of the portable operations device shown in FIGS. 1-4;

FIG. 10 is a schematic view of the handheld display device shown in FIGS. 1-4;

FIG. 11a is a perspective front view of the an amplifier according to the present invention shown worn within a holster;

FIG. 11b is a perspective side view of the holster shown in FIG. 11a; and

FIG. 12 is a perspective front view of an amplifier belt according to a preferred embodiment of the present invention.

### DETAILED DESCRIPTION OF THE PREFERRED EMBODIMENTS

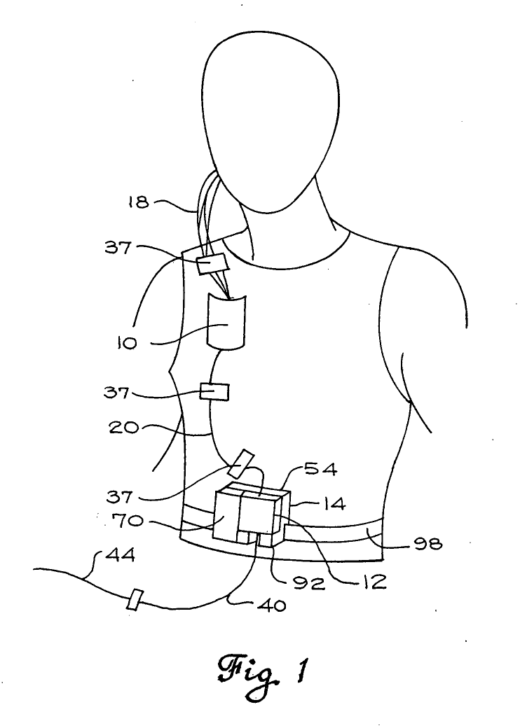

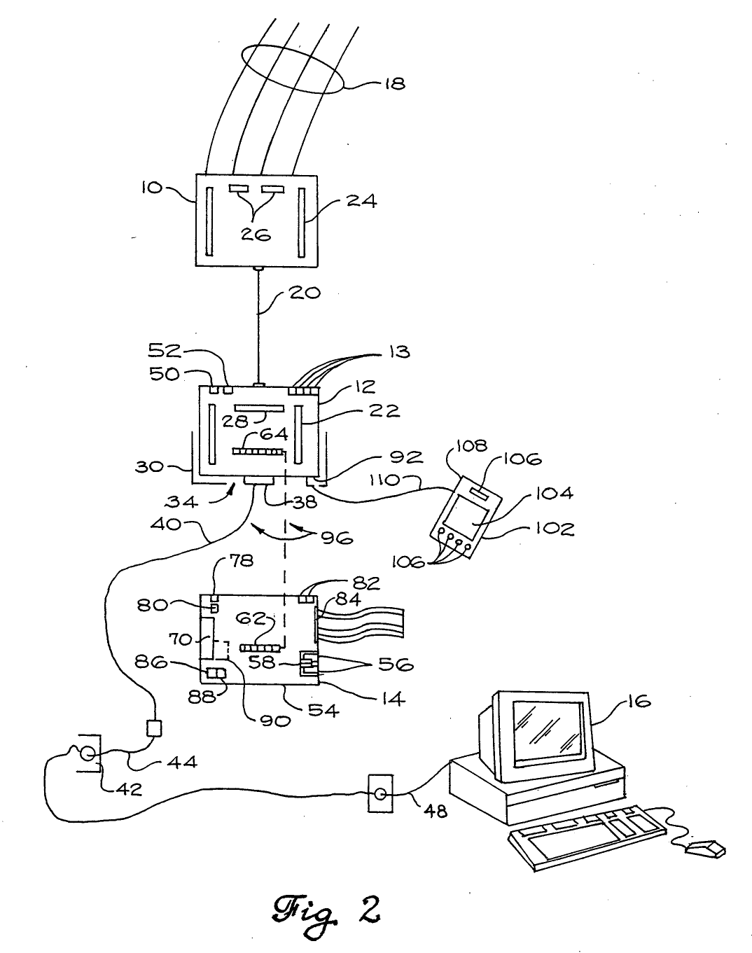

With reference to FIGS. 1 and 2, a preferred embodiment of the present invention employs a jackbox 10, an amplifier 12, and a portable operations device (POD) 14 in communication with a host computer 16. The jackbox 10, amplifier 12, and portable operations device 14 can preferably be worn by or otherwise carried upon a patient. The jackbox 10 can be of any conventional type, and has a plurality of electrode connectors (not shown) for connection to a plurality of conventional electrodes 18. The electrodes 18 can be surface, subdermal, depth, or other types of electrodes, and can be arranged on the patient in any manner desired, such as in particular locations on the patient's head, in a grid or array, and the like. If desired, a combination of different electrode types and manners of connection to the patient can be employed.

The host computer 16 can be any type of computer device or system capable of processing patient physiological signals and data, including in some preferred embodiments digital video data and textual data, received from the amplifier 12. The host computer 16 also is capable of either displaying or storing such signals and data or transmitting such data to another device or system for such purposes. For example, the host computer 16 can be any type of personal computer (PC) that is stand-alone, mobile, or is connected to a network of other computers, can be a mainframe computer system, and the like.

The jackbox 10 can be any size and can be adapted to connect to any number of electrodes 18 in manners well known to those skilled in the art. By way of example only, the jackbox 10 can have 32 electrode connections (e.g., shielded male connectors sized to mate with female connectors on the ends of the electrodes 18, female connectors sized to mate with male connectors on the ends of the electrodes 18, and the like) of which any number are to be connected to reference electrodes, electrodes for single-polar channels, and electrodes for bi-polar channels. All three types of electrodes and their manner of operation and connected to a patient are well known to those skilled in the art and are not therefore described further herein. In one highly preferred embodiment, the 32 electrode connections provide 4 bi-polar channel connections and have 24 electrodes connected to one or more of 4 reference electrodes. Any other number of reference electrodes and electrodes providing uni-polar and bi-polar channels can be used, each such combination falling within the spirit and scope of the present invention.

A 68-pin cable 20 preferably electrically connects the jackbox 10 to the amplifier 12. Conventional communications jacks or ports on the jackbox 10 and amplifier 12 are preferably used to connect the cable 20 thereto. However, any conventional cable 20 capable of transmitting the signals received from the various jackbox electrode connectors can be employed. In highly preferred embodiments of the present invention, the cable 20 is shielded against electromagnetic interference. Depending at least in part upon the jackbox electrode connection capacity, the cable can have a greater or lesser pin count.

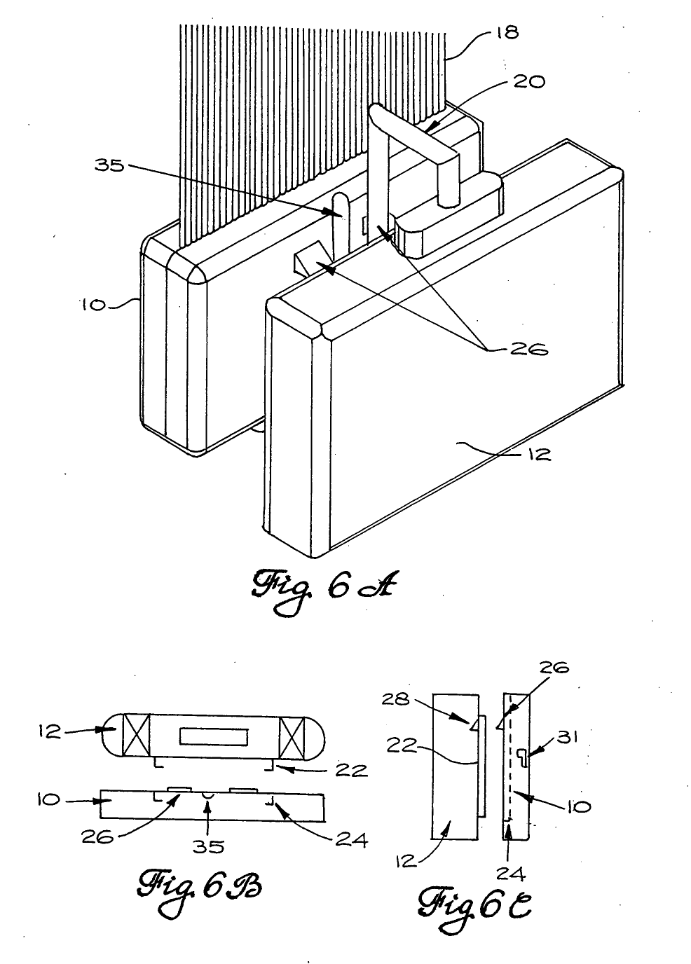

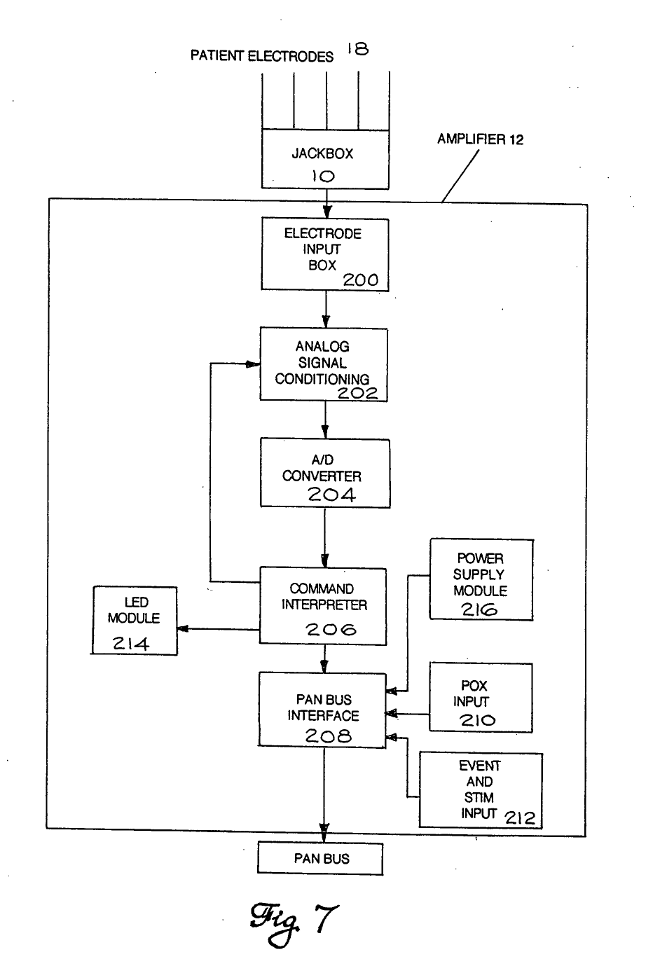

The jackbox 10 can be located a distance from the amplifier 12 as shown in FIG. 1, or can be connected to the amplifier 12 in a manner as shown in FIGS. 6a-6c. At least in such applications where the jackbox 10 is mountable upon the amplifier 12, the jackbox 10 and amplifier 12 preferably have interconnecting elements which can be releasably engaged to connect the jackbox 10 and amplifier 12 together. In some highly preferred embodiments, these elements are mating rails and tracks and mating barbs and detents. With continued reference to FIGS. 6a-6c for example, rails 22 on the amplifier 12 are preferably received within tracks 24 in the jackbox 10, whereby the jackbox and amplifier are slid relative to one another with the rails 22 in the tracks 24. Upon reaching a desired position with respect to one another, detents 26 in the jackbox 10 preferably mate with one or more apertures or recesses 28 in the amplifier 12, thereby securing the jackbox 10 in place upon the amplifier 12. Resiliently deformable elements such as springs, elastomeric pads, and the like can be used to urge the detents into mating engagement with the apertures or recesses 28. The jackbox and amplifier connection can be released in a number of conventional manners, such as by a release button, slide, or lever 31 on the jackbox 10 connected to the detents 26 to retract the detents 26 from the amplifier recess 28. Such releasable detent mechanisms are well known to those skilled in the art and are not therefore described further herein.

When the jackbox 10 is mounted upon the amplifier 12, the cable 20 connecting these two devices can be routed in any manner desired. The communications jacks or ports on the jackbox 10 and amplifier 12 to which the cable 20 is connected can be located at any locations on these devices, but preferably are located on the bottom of the jackbox 10 and the top of the amplifier 12 as shown in FIGS. 6a-6c. The cable 20 in such an arrangement is preferably routed between the jackbox 10 and amplifier 12 (in a recess or groove 35 in one or both devices, if desired) to provide a compact assembly and to reduce the amount of unsecured cable 20. Any other manner of routing the cable 20 can instead be used as desired.

Any number of detents 26 and recesses 28 (even none) can be used to secure the jackbox 10 and amplifier 12 together, and any number of rails 22 and tracks 24 (even none) can be used to orient and/or guide the jackbox 10 relative to the amplifier 12. In addition, the detent and recess connection can be replaced by any number of different inter-engaging elements performing the same functions, including without limitation aperture and pin connections, latch and aperture connections, post and catch assemblies, buckles, clasps, aperture and ball bearing assemblies, dimple or rib and recess sets, engaging resilient pawl and recess sets, and the like. Such alternative securing elements are well-known to those skilled in the art and fall within the spirit and scope of the present invention. Also, the rails and track connections can be replaced by any number of different guiding and positioning elements, including without limitation pin and track assemblies, mating grooves and ribs on the surfaces of the jackbox 10 and amplifier 12, and the like. Such alternative guiding and positioning elements are also well-known to those skilled in the art and fall within the spirit and scope of the present invention.

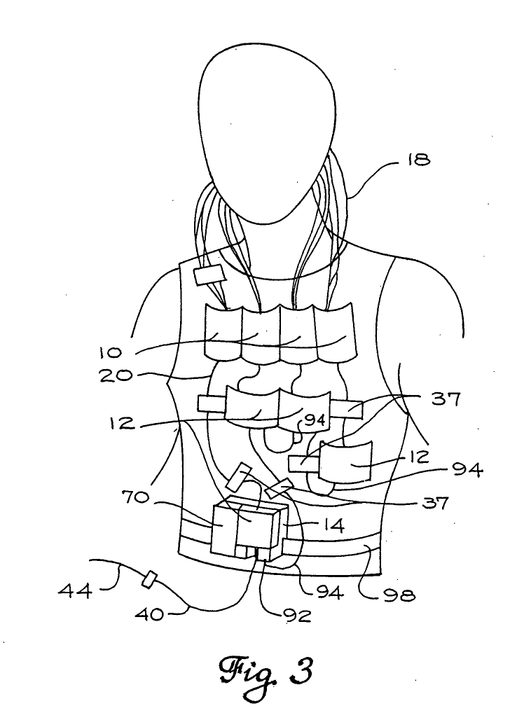

As described above, the jackbox 10 in some highly preferred embodiments is not mounted upon the amplifier 12 or can be either mounted on the amplifier 12 or located a distance therefrom as desired. Where the jackbox 10 is not mounted on the amplifier, the jackbox 10 can preferably be secured to the patient in a number of conventional manners, including without limitation by being received within a garment pocket or pouch (e.g., in a vest having such an internal or external pocket or pouch), by hook and loop fastener elements on the jackbox 10 and on a garment or other element worn by the patient, by a cuff, belt, harness, strap, holster, or other device worn by the patient, by adhesive strips, pads, or other elements, by one or more clasps, clips, snaps, ties, or other conventional fasteners attached to a garment or other article worn by the patient, and the like. Alternatively or in addition, the jackbox 10 can be held upon the patient by fastening elements 37 holding the electrodes 18 and/or cable 20 connected to the jackbox 10 as shown in FIGS. 1 and 3. For example, hook and loop fastener strips, ties, adhesive tape, clips, clasps, strips with snaps, or other conventional fastening elements can be used to hold the electrodes 18 and cable 20 on either side of the jackbox 10 to a garment or article worn by the patient.

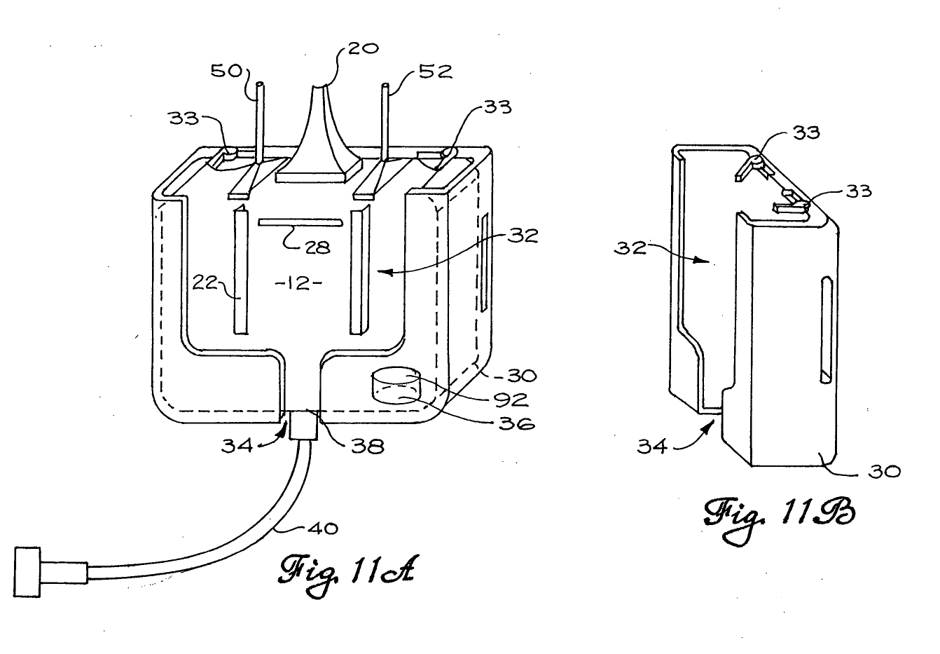

The amplifier 12 can be held to or secured upon the patient in any of the manners described above with reference to the jackbox 10, but preferably is held within a holster 30 as shown in FIGS. 11a and 11b. The holster 30 can be made of any material desired, such as plastic, metal, urethane, leather, and the like, but preferably is made of resilient plastic. The holster 30 can be any shape to surround any amount of the amplifier 12, but preferably has an open front area 32 for connection to the jackbox 10 (if desired) leading to an open bottom area 34 for cabling connection (also if desired) as discussed in greater detail below. In some highly preferred embodiments of the present invention, the holster 30 also has at least one slot, notch, opening, or other aperture 36 for additional cabling connection to the amplifier 12. The holster 30 can be held to or secured upon the patient in any of the manners described above with reference to the jackbox 10. Most preferably, the holster 30 is secured to patient via a belt passed through one or more loop holes in the holster 30 as seen in FIGS. 11a and 11b.

The amplifier 12 is preferably retained in the holster 30 by one or more latches 33. In the illustrated preferred embodiment, the latches 33 are on the holster 30 can preferably be pivoted to positions preventing removal of the amplifier 12. In alternate embodiments, the latches 33 can be on the amplifier 12 and can be movable into apertures, notches, or recesses in the holster 30 for the same purpose. It should be noted that a number of conventional manners exist for releasably retaining the amplifier 12 in the holster 30, including without limitation any of the manners of connection described above with reference to the jackbox-to-amplifier connection, by form-fitting resiliently deformable portions of the holster 30 to the amplifier 12, by one or more conventional fasteners, by belt loop holes on the amplifier 12, by one or more straps, hinged arms or doors, hook and loop fastener material on the holster 30 and amplifier 12, and the like.

The jackbox 10 and amplifier 12 represent an assembly that can be directly connected to the host computer 16 by a communications cable and/or by wireless transmission in a conventional manner. To this end, the amplifier 12 can have a communications jack or port 38 for communication with the host computer 16 via a cable 40 connected to the jack or port 38. The cable 40 can be of any conventional type, but most preferably is a 8-pin high speed cable.

The manner in which the jackbox 10 and amplifier 12 are connected via communications cabling to a host computer 16 can vary significantly from application to application. For example, the cable 40 extending from the amplifier 12 can connect directly to the host computer 16 (with conventional adapter cabling or adapter devices if necessary), in which case user connection and disconnection of the tether cable 40 can be made at the amplifier jack or port 38 and/or at the host computer 16. In other applications such as where the patient and host computer are in different rooms or locations, the cable 40 can extend to a wall jack or port 42 which is electrically connected to the host computer 16 via an in-wall communications cable 46 connected either directly to the host computer 16 or via an extension or adapter communications cable 48 to the host computer 16. Although possible, connection and disconnection is preferably not made at the host computer 16 or at the amplifier 12. Instead, the cable 40 extending from the amplifier 16 is preferably connected to an extension cable 44 running to the host computer 16 or to the wall jack or port 42. An extension cable 44 provides easy and accessible patient connection and disconnection between the cable 40 running from the amplifier 12 and the wall jack or port 42. Preferably, the mating connectors of these cables provides a locking connection in any conventional manner to prevent inadvertent patient disconnection. The cable running to the host computer 16 can connect thereto in any conventional manner, and preferably connects in a conventional manner directly to a communications jack or port on an interface card in the host computer 16.

The cabling running between the host computer 16 and the amplifier 12 preferably transmits power to the amplifier 12 in addition to transmitting patient physiological signals and communications signals. Although a number of conventional computer networking systems are capable of transmitting power and signals, the present invention preferably employs a peripheral area network (PAN) specifically configured for the present invention for physiological and communications signal control and routing and for supplying power to the amplifier 12 and the jackbox 10 from the tethered power supply.

As an alternative to transmitting signals via cable as described above, the amplifier 12, via the portable operations device 14 described below, can instead transmit the signals with a conventional wireless transmitter. A conventional wireless transmitter is capable of transmitting the signals as infrared, microwave, or any other conventional frequency signals. The signals are then received by a conventional wireless receiver connected to or in the host computer 16. In addition or instead, the amplifier 12 and the host computer 16 can be provided with a conventional wireless receiver and a conventional wireless transmitter, respectively, for sending signals to the amplifier 12 as desired. Wireless transmitters and receivers, their connection, and their manner of operation are well known to those skilled in the art and are not therefore described further herein. However, in some preferred embodiments where wireless transmitters are employed to transmit physiological signals, control signals, compressed video, and/or compressed audio, as described above, the transmissions are preferably spread spectrum transmissions processed and transmitted in any conventional manner. In embodiments of the present invention employing wireless signal transmission directly between the amplifier 12 and the host computer 16, power can be provided to the amplifier 12 by a power cord or by a battery.

In addition to amplifying physiological signals transmitted from the jackbox 10, preferred embodiments of the present invention employ an amplifier 12 having additional jacks or ports for receiving physiological signals and/or other patient monitoring signals. For example, the amplifier 12 preferably has an input 50 for connection to a pulse oximeter (not shown) and an input 52 for connection to an event marker pendent (also not shown). The pulse oximeter is conventional in nature and operation, and provides the amplifier with signals indicating the oxygen saturation of the arterial blood supply. The event marker pendent is also conventional in nature and operation, and is preferably a handheld device for the patient. The event marker pendent has a patient-manipulable control such as a button, switch, lever, and the like which can be triggered by the patient to send a signal to the amplifier to note the occurrence of an event (such as the onset of pain, a particular sensation, etc.). The event marker pendent can take a number of other forms that may or may not be handheld, but in each case preferably provides a patient-manipulable control for the above-noted purpose. Still other jacks or ports can be located on the amplifier 12 for receiving other physiological signals and/or patient monitoring signals. Such jacks or ports can be used for the connection of a motion sensor, a microphone, video images, a body position sensor, a blood pressure and/or pulse monitoring device, a body temperature sensor, a breathing monitoring device, or any other patient monitoring apparatus producing signals representative of patient physiological activities. As will be described in more detail below, such other jacks and ports are more preferably located on the portable operations device 14.

With reference to FIG. 7, the amplifier 12 preferably interfaces with the patient electrodes 18, converts the electrical signals into digital data, and transmits that information to the host computer 16. The amplifier 12 most preferably comprises nine subsystems although any number of subsystems can be used satisfactorily. These subsystems preferably comprise an electrode input block 200, an analog signal conditioning section 202, an analog to digital (A/D) converter 204, a controller (preferably a command interpreter 206), a peripheral area network bus interface 208, a pulse oximeter (POX) input 210, an event pendent and stimulation input 212, a light-emitting diode (LED) module 214, and a power supply module 216.

The electrode input block 200 connects the electrode inputs from the jackbox 10 to the amplifier 12. In one preferred embodiment, the electrode input block 200 is in the form of a 68 pin memory data register (MDR) type connector. The 68 pin connector provides inputs for 32 active electrodes that support referential monitoring, four switchable reference electrodes, eight active electrodes for a total of four dedicated bipolar channels, an isolated patient ground, two jackbox identification pins, and two electrode group identification pins. An analog switching matrix in the electrode input block 200 connects the reference electrodes either individually or in groups to the referential amplifier channels. The jackbox identification pins are routed to a programmable logic device (PLD) called a field programmable gate array (FPGA) that is part of the command interpreter 206 for storage. A FPGA is a logic integrated circuit consisting of interconnectable gates. The interconnection of the gates determines the functionality of the FPGA. The interconnection is programmable via software and is more flexible to change than other PLDs.

The analog signal conditioning section 202 connects the electrode input block 200 to the A/D converter 204. In one preferred embodiment, the analog signal conditioning section 202 comprises a set of input instrumentation amplifiers, a 36-1 multiplexer, and various analog filters, all of which are well know to those skilled in the art.

The analog signal conditioning section 202 most preferably includes input instrumentation amplifiers for the 32 referential channels and for the four bipolar channels. With the amplifiers, the analog signal conditioning section 202 provides independent gain control on a channel-by-channel basis. The amplifiers are chosen to meet size and power requirements in addition to common mode rejection ratio (CMRR) requirements. The analog signal conditioning section 202 provides blocking capacitors to remove the DC component of the signal. The input signals feed into the blocking capacitors, which feed into the multiplexer, which feeds into a resistor, for a highly integrated implementation. The analog signals are transmitted via the multiplexer to the resistor which has the effect of multiplying the resistance by 36, hence acting as a high pass filter by effecting the cutoff frequency. The FPGA generates the multiplexer clock signal, which coordinates the receipt of the signals by the A/D converter 204 from the amplifier 12. The analog signal conditioning section 202 includes either a two-pole or, most preferably, a three-pole Butterworth low-pass filter with a roll off at 500 Hz. The op amps for the low pass filter are designed to meet size and power constraints in addition to offset and noise requirements. The analog signal conditioning section 202 also facilitates a Deblock function, which is well known to those skilled in the art. An analog switch shorts the blocking capacitors to ground in order to implement the Deblock function. The output of the analog signal conditioning section is a multiplexed analog time sample that is 5 microseconds long, such that a single A/D converter 204 may be used.

The A/D converter 204 provides synchronous 16-bit digitization, although higher synchronous digitization is possible. In one preferred embodiment, the A/D converter 204 operates at a speed sufficient to hold interchannel time skew to a worst case level of less than or equal to 500 microseconds, preferably over 128 channels. Additionally, the A/D converter 204 serializes the data for transmission on the peripheral area network bus. An A/D convert pulse is generated by the FPGA and is triggered by a command from the peripheral area network bus.

In one preferred embodiment, the command interpreter 206 comprises a microprocessor and a portion of the FPGA. Alternatively, the command interpreter 206 may be an application specific integrated circuit (ASIC), or a combination of a microprocessor and an ASIC other than a FPGA. The command interpreter 206 switches front end reference signals. The command interpreter 206 receives and decodes both pulse oximeter (POX) data and event input button data, and then provides the data to the FPGA for synchronous transmission with the EEG data on the peripheral area network bus. The command interpreter 206 receives the jack box 10 identification and electrode group identification for further processing. The command interpreter 206 also lists the channel by channel gains, stores the non-volatile parameters such as the amplifier identification, and pulses a watchdog timer.

The peripheral area network bus interface 208 acts as an input/output (I/O) interface for the command interpreter 206. The peripheral area network bus interface 208 receives data and commands from the peripheral area network bus, formats the data and commands, and provides the data and commands to the command interpreter 206. In one preferred embodiment, the peripheral area network bus interface 208 is implemented in the FPGA. The FPGA receives data from the A/D converter 204 and transmits it to the peripheral area network bus as requested. Additionally, the FPGA receives, stores, and transmits the address of the amplifier on the peripheral area network bus to the microprocessor, via a read-only register. The FPGA then generates an output address based on the input address of the amplifier module.

The pulse oximeter input 210 is a connector that supplies power from the amplifier to and receives data from the SpO₂ detection circuitry. In one preferred embodiment, the microprocessor and the FPGA both receive pulse oximeter data, while only the microprocessor processes the pulse oximeter data. The SpO₂ data is eventually transmitted to the host interface card via the peripheral area network bus.

The event and stimulation input 212 connects an event marker pendent input to the amplifier 12. In one preferred embodiment, the command interpreter 206 receives the event marker input. When the command interpreter 206 receives an event marker input, the microprocessor of command interpreter 206 provides this information to the FPGA, and the FPGA transmits the information to the host interface card 220 via the peripheral area network bus. The host interface card 220 then marks the incoming data as being associated with the occurrence of a patient event.

The event and stimulation input 212 also connects a stimulation input to the amplifier 12. Preferably, the command interpreter 206 receives the stimulation input. In one preferred embodiment, the same electrodes that are used for acquiring patient signals are also used for cortical stimulation. When the same electrodes are used for both acquisition and stimulation, the electrodes must begin acquiring data immediately after the stimulation signal is transmitted to the patient in order to accurately monitor the patient's reaction to the stimulation signal. When the command interpreter 206 receives a stimulation input, the input is processed rapidly by the FPGA. The FPGA rapidly disconnects the stimulation current flow to the patient electrodes from the amplifier inputs. The FPGA then initiates the Deblock function to short the capacitors in the amplifier circuit to ground. With the Deblock function, the amplifier 12 does not saturate and goes to zero baseline instantly. After stimulation is completed, the amplifier 12 returns to normal operation. In this manner, the FPGA isolates the electrical inputs from the internal circuitry, such that the energy associated with the cortical stimulator does not saturate the amplifiers or damage the electronics. Thus, the FPGA allows the amplifier channels to pass only those biopotentials generated in response to the stimulus energy. In another preferred embodiment, a photic stimulator controller flashes a strobe light into the patient's eyes at different frequencies. The stimulation input to the amplifier 12 marks the acquired data as a response to the photic stimulation.

The LED module 214 indicates the state of the amplifier or any error conditions. In order to provide the user with information regarding the operation and/or status of the apparatus, the amplifier 12 can have one or more indicators. These indicators are preferably LEDs, although any light, display, or other visual or audible indicator can instead be used. In the illustrated preferred embodiment for example, the amplifier 12 is provided with four LEDs 13: one to indicate that the electrodes 18 are being calibrated, another to indicate that the electrodes are being tested or that the Deblock function is in process, another to indicate that the amplifier has been disconnected from the jackbox 10, and another to indicate that the monitoring apparatus is running. In other embodiments of the present invention, the amplifier 12 can have more or fewer indicators providing some or all of this information to the user. Preferably, the indicators 13 on the amplifier 12 are located in a position on the amplifier that is not covered or otherwise obstructed from user view when the amplifier 12 is placed in a holster, is secured to or held within any other garment or wearable element as described above, has the jackbox 10 mounted thereon as also described above, or is connected to the portable operations device 14. In the illustrated preferred embodiment for example, the indicators 13 are located on the top of the amplifier 12. In one preferred embodiment, the microprocessor sets the LEDs to the appropriate configuration, based on the subsystem status, via the FPGA control registers that drive the LEDs directly.

The power supply module 216 provides power to the components as required, while providing type CF isolation where necessary. In one preferred embodiment, the power supply module 216 is controlled by the microprocessor and the FPGA. The FPGA provides bits in the mode control register to enable and disable power to various parts of the circuit. The FPGA also directs isolated power to the appropriate circuits when required.

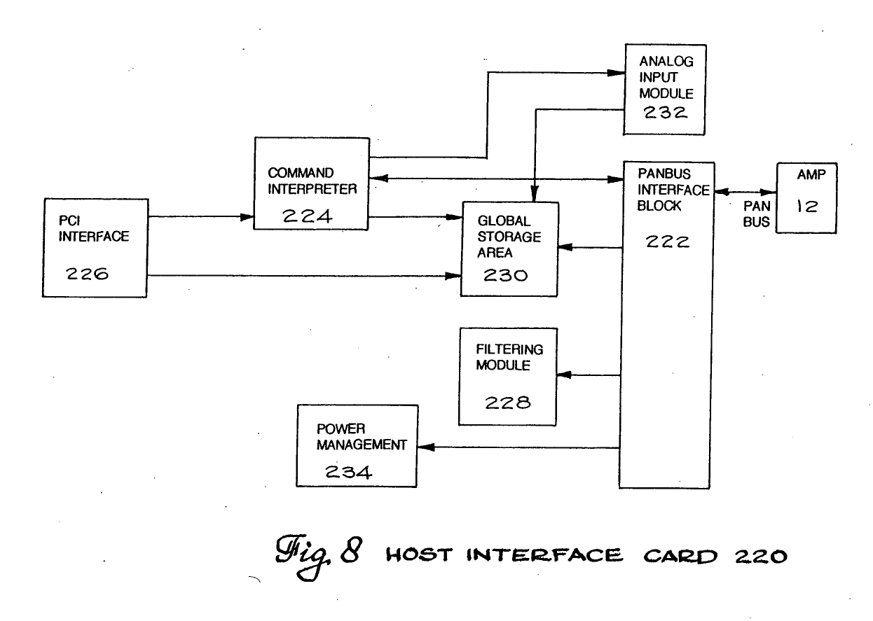

With reference to FIG. 8, the host interface card 220 is responsible for receiving data from the amplifier 12 and providing it to the host computer 16. When the patient is tethered to the host computer 16, the host interface card 220 is used to communicate the data from the amplifier 12 to the host computer 16. In one preferred embodiment, the host interface card 220 comprises seven components, including a peripheral area network bus interface 222, a controller (preferably a command interpreter 224), a peripheral component interconnect (PCI) interface 226, a filtering module 228, a global storage area 230, an analog input module 232, and a power management section 234.

The peripheral area network bus interface 222 receives data from the peripheral area network bus and routes the data appropriately, depending on the data's configuration. The peripheral area network bus interface 222 formats and outputs commands from the command interpreter to the peripheral area network bus. The peripheral area network bus interface 222 receives, stores, and transmits the address of the amplifier on the peripheral area network bus to the command interpreter. Additionally, the peripheral area network bus interface 222 provides power to all the devices on the peripheral area network bus. In one preferred embodiment, the peripheral area network bus interface 222 is implemented by a FPGA. The peripheral area network bus interface 222 receives power from the power management section 234 and provides this power to other devices on the bus.

The command interpreter 224 is implemented by an Intel 8052 microprocessor in one highly preferred embodiment, although other microprocessors or circuitry can be used without departing from the invention. The command interpreter 224 formats and transmits commands created by the host computer and intended for the peripherals on the peripheral area network bus. The command interpreter 224 receives push button inputs from a handheld display unit 102 and converts push button inputs into requests for action by the host computer 16. The command interpreter 224 communicates with the portable operations device 14 for bus mastering transfer. The command interpreter 224 controls the state of the host interface card 220 when peripherals are removed from the peripheral area network bus while being run by the portable operations device 14. The command interpreter 224 calculates the threshold impedance value for the patient electrodes 18, subsamples the data, and maintains electrode name lists. The command interpreter sends the CAL and ETEST waveform data to the peripheral area network bus interface 222 for transmission to the handheld display unit 102. The command interpreter sets the gains of the analog input module 232. The command interpreter 224 controls the optional wireless LAN and controls a watchdog timer in case of errant processor behavior.

The PCI interface 226 is responsible for PCI bus interactions. The PCI interface 226 allows the host computer's PCI bus to read from and write to the global storage area 230. The PCI interface 226 passes host computer 16 messages to the command interpreter 224. Additionally, the PCI interface 226 implements all requirements to support Plug-n-Play. In one preferred embodiment, the PCI interface is implemented by a PCI bridge integrated circuit, PLX9050.

The filtering module 228 performs low-pass, high-pass, band-elimination, and downsampling filtering. In various alternative embodiments, the filtering module provides a first order high-pass filter configurable to one of the following cutoff frequencies: 0.1, 0.3, 0.5, 1.0, 1.5, 2.0, 2.5, 5.0, and 10.0 Hz. The filtering module 228 provides a sixth order infinite impulse response (IIR) low-pass filter configurable to one of the following cutoff frequencies: 500, 150, 100, 70 Hz. In alternative embodiments, the low-pass filter could be implemented as a finite impulse response (FR) or decimation filter, as long as the stopband attenuation is equivalent to a 6^{th} order IIR filter. The filtering module 228 provides band elimination for the removal of line noise and is configurable for either 50 or 60 Hz. In one highly preferred embodiment, the filtering module is implemented by a TMS320C44 digital signal processor (DSP); however, the filtering module can be implemented by other digital signal processors. It will be apparent to one of ordinary skill in the art that discrete modules can be used to perform the filtering functions of the filtering module 228 without departing from the invention.

The global storage area 230 receives raw data from the peripheral area network bus and digitized data from the analog inputs. The global storage 230 area buffers the peripheral area network bus and ring buffers the SpO₂ data. The global storage area 230 provides arbitration between the devices wishing to access the memory, including the microprocessor, the filtering module 228, the analog input module 232, and the PCI interface 226. In one preferred embodiment, the global storage area 230 is a large block of RAM which is accessible to all devices through the FPGA.

The analog input module 232 provides for 32 additional 0V to 1V high-level inputs in one preferred embodiment. The analog input module 232 provides unity gain amplifiers. The analog input module 232 provides a 150 Hz analog single-pole low-pass filter, comprised of one resistor and one capacitor, for each channel. The analog input module 232 has a bandwidth from DC to 150 Hz. In one preferred embodiment, the analog input module 232 is implemented by a DB44 HD connector and instrumentational amplifiers set for unity gain. In addition to the unity gain amplifiers, the gain for each individual channel can be controlled by the command interpreter 224. The gain factors for each individual channel are x1, x10, and x100. The gain factors are implemented by analog switches, which switch in one of four appropriate resistor networks to provide the four gains. Once amplified and filtered, the host interface card A/D converter (not shown) receives all 32 inputs via a multiplexer. Preferably, the data is digitized at 2000 samples per second and is provided to the global storage area 230 for further processing.

The power management section 234 provides power to the peripheral area network bus interface 222 for distribution to all of the devices on the peripheral area network bus. In one preferred embodiment, the power management section 234 receives feedback in order to compensate for voltage drops over long cable runs. A voltage sense line at the end of the tether sends feedback to the power management section 234. The power management section 234 adjusts the voltage provided to the peripheral area network bus interface according to the feedback provided, such that the voltage provided to the devices on the peripheral area network bus remains constant. The power management section 234 is implemented with a DC to DC converter feedback circuit under analog control.

Although the jackbox 10 and amplifier 12 can be tethered or wirelessly connected to the host computer 16 as described above, it is often desirable to increase patient mobility and to increase the quality of data acquisition and storage during patient monitoring. For these and other purposes to be described below, the jackbox 10 and amplifier 12 can be connected to the portable operations device 14. The portable operations device 14 has a housing 54, at least one bay 56 in the housing 54 for removably receiving a peripheral card 58, and the command interpreter 206 for routing signals received from the amplifier 12 to the bay(s) 56 as will be described in more detail below. The portable operations device 14 has a communications jack or port 62 for connection to a communications jack or port 64 of the amplifier 12. The communications jack or port 64 on the amplifier 12 is preferably different than the above-described communications jack or input 38 on the amplifier 12 connected directly via cable(s) 40, 44, 46, 48 to the host computer 16. Although the communications jack or port 64 on the amplifier 12 can be connected to the communications jack or port 62 on the portable operations device 14 by a cable or even by wireless transmission in a manner similar to the above-described connection between the amplifier 12 and the host computer 16, this connection is more preferably established by insertion of the amplifier 12 in the portable operations device 14. Specifically, the communications ports 62, 64 are preferably mating terminals on the portable operations device 14 and amplifier 12, respectively, and electrically connect when the amplifier 12 is inserted into the portable operations device 14. The communications ports 62, 64 and their manner of connection are conventional in nature and are not therefore described further herein.

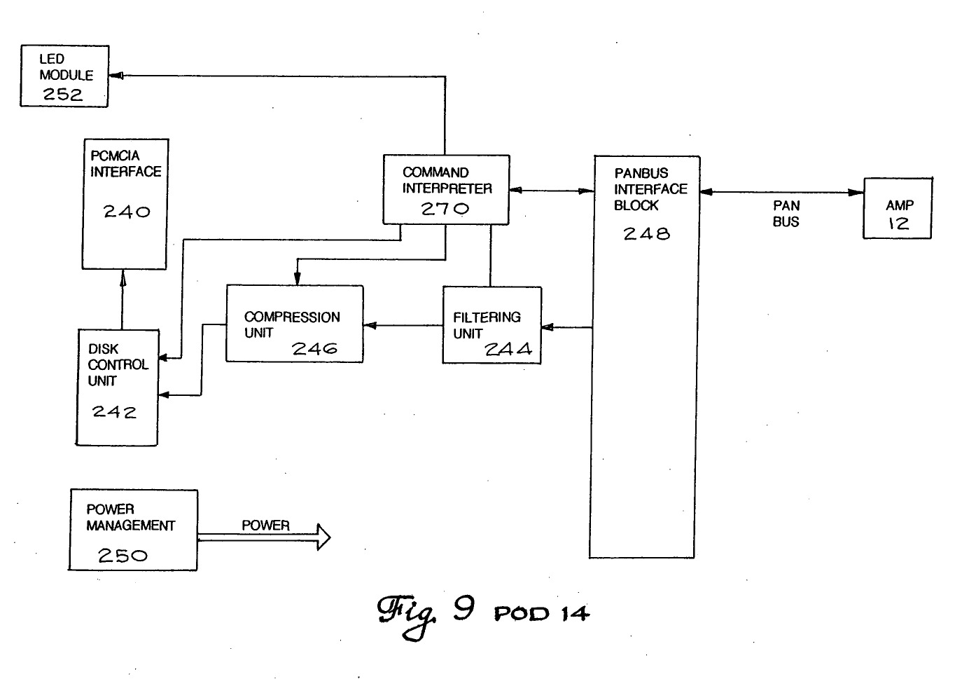

With reference to FIG. 9, the portable operations device 14 is responsible for the storage of data, as well as bus control when the host interface card 220 is not present. When the patient is untethered from the host computer 16, the portable operations device 14 is used to communicate the data from the amplifier 12 to a storage device located within the portable operations device 14. In addition to storing the data, the portable operations device 14 is capable of performing all the primary functions of the host interface card 220. Alternatively to storing the data, the portable operations device 14 is also capable of transmitting the amplifier data to the host computer 16 or another base unit via wireless LAN. The portable operations device 14 is capable of transmitting compressed video data from the amplifier 12 to the host computer 16 or another base unit via wireless LAN. One highly preferred embodiment of the portable operations device comprises eight components, including a Personal Computer Memory Card International Association (PCMCIA) interface 240, a disk control unit 242, the command interpreter 270, a filtering unit 244, a compression unit 246, a peripheral area network bus interface module 248, a power management module 250, and an LED module 252.

The PCMCIA interface 240 is preferably comprised of at least one bay 56. In one highly preferred embodiment, the portable operations device has two bays 56. In some highly preferred embodiments of the present invention, the bay(s) 56 of the portable operations device 14 meet the PC card standards of the Personal Computer Memory Card International Association, and therefore accept PCMCIA-type peripheral cards. Most preferably, the portable operations device 14 has a PCMCIA bay 56 capable of accepting two PCMCIA Type II cards or one PCMCIA Type III card. PCMCIA cards with several different combinations of functionality are available, such as RAM/RAM, RAM/disk, wire/wireless, wire/RAM, wire/disk, wire/modem, or RAM/modem, wire/network, RAM/network, and network/modem. However, the bay 56 can instead be adapted in a well-known manner to receive any number of such cards as desired. Most preferably, the peripheral card or cards 58 are memory media such as rotating disk media, memory chips, modems, and the like, and are removably insertable into the bay 56 to receive and store patient physiological signal data. In one preferred embodiment, a PCMCIA wireless transmitter peripheral card 58 can instead be inserted into the bay 56 for receiving such physiological signal data and wirelessly transmitting the data to the host computer 16. In another preferred embodiment, the bay 56 receives at least one PCMCIA memory card and one PCMCIA wireless transmitter card. In still another embodiment of the present invention, the PCMCIA interface 240 includes video ports.

One having ordinary skill in the art will appreciate that the bay and peripheral cards employed in the present invention need not necessarily be PCMCIA standard elements, and that any conventional bay and card standard or type can instead be used. Also, although the peripheral cards 58 are preferably removable from the portable operations device 14 by the user, this is not a requirement of the present invention. Alternatively, the portable operations device 14 can have on-board re-writeable memory of any conventional type not intended for regular user replacement during normal operation of the apparatus.

The disk control unit 242 properly formats the data from the amplifier for whichever storage or transmitting device is installed in the bay 56. In various preferred embodiments, the disk control unit 242 properly formats compressed video data, algorithms, and the activation of devices, such as a stimulator. The disk control unit 242 also is retrieves any required software updates to the command interpreter 270 for installation. In one highly preferred embodiment, the disk control unit 242 is implemented by the microprocessor of command interpreter 270 with additional support circuitry to provide the electrical interface between the microprocessor and the PCMCIA bay.

The command interpreter 270 is responsible for receiving data, preferably data including compressed video and audio, and executing commands from the peripheral area network bus. The command interpreter 270 configures the other components of the portable operations device 14 and initializes the portable operations device 14 in case of power loss. The command interpreter 270 oversees the movement of formatted data to the disk control unit 242. The command interpreter communicates with the host interface card 220 when the patient is tethered to the host computer 16 to effect a bus master transfer. The command interpreter updates software via the PCMCIA interface 240 and provides watchdog functionality for the filtering unit 244. In one highly preferred embodiment, the command interpreter is implemented using an Intel 1110 StrongArm microprocessor and an Altera 6024A FPGA, both of which are located within the portable operations device 14.

The filtering unit 244 performs low-pass, high-pass, band-elimination, and downsampling filtering. In various alternative embodiments, the filtering unit 244 provides a first order high-pass filter configurable to one of the following cutoff frequencies: 0.1, 0.3, 0.5, 1.0, 1.5, 2.0, 2.5, 5.0, and 10.0 Hz. In one preferred embodiment, the filtering unit 244 provides a sixth order IIR low-pass filter configurable to a cutoff frequency of either 500 Hz or 100 Hz. In another preferred embodiment for monitoring electromyography (EMG) signals, fewer channels of data are gathered with a higher bandwidth of approximately 3000 Hz to 10 kHz. In alternative embodiments, the low-pass filter could be implemented as a FR or decimation filter, as long as the stopband attenuation is equivalent to a 6^{th} order IIR filter. The filtering unit 244 provides band elimination for the removal of line noise and is configurable for either 50 or 60 Hz. The filtering unit 244 provides downsampling filtering, which ensures that sufficient low-pass filtering is in place such that aliasing is at least minus 12 dB down. In one preferred embodiment, the filtering unit 244 is a TMS320C5416 digital signal processor that receives data in time-division multiplexing (TDM) format from the FPGA via multi-channel buffered serial ports (McBSP) and transfers the data to internal memory.

The compression unit 246 compresses incoming data from the amplifier before it is transmitted or stored on the PCMCIA storage device. The compression does not cause any clinically significant changes to the data after decompression. The compression unit 246 is implemented in either the microprocessor of command interpreter 270 or the digital signal processor of filtering unit 244.

In another preferred embodiment, the portable operations device 14 does not include the filtering unit 244 or the compression unit 246. Rather, the portable operations device 14 simply receives the data and stores or transmits the data via the PCMCIA interface 240 or the like. As PCMCIA devices and other storage devices become larger and more cost effective, the need for filtering and compressing data may diminish.

The peripheral area network bus interface module 248 receives data and commands from the peripheral area network bus and provides the data and commands to the command interpreter 270. The peripheral area network bus interface module 248 also formats outgoing responses and other information from the command interpreter 270 to the peripheral area network bus. Additionally, the peripheral area network bus interface module 248 receives, stores, and transmits the address of the portable operations device on the peripheral area network bus to the command interpreter. The peripheral area network bus interface module 248 then generates an output address based on the input address of the portable operations device. In one preferred embodiment, the peripheral area network bus interface module 248 is implemented by a FPGA.

The power management module 250 provides power to all the components in the portable operations device 14. The power management module 259 ensures that minimum power consumption is attained by implementing power-down modes for all the components. When the patient is untethered from the host computer 16, the power management module 250 also provides power to the peripheral area network bus interface 248 when directed to do so by the command interpreter 270 and is capable of doing so for a total of 26 hours in one preferred embodiment. In one preferred embodiment, the microprocessor is responsible for management of the power for the power-down modes in all the components. The microprocessor uses registers in the FPGA to control the power. Additionally, the digital signal processor is programmed to help minimize power consumption by taking full advantage of any possible low-power modes. The power management module 250 can use a 7.2V Lithium battery, although other batteries and power sources can be used.

The LED module 252 displays the status of the portable operations device 14. To provide information to the user regarding the operation of the portable operations device 14, the portable operations device 14 can have one or more indicators thereon. These indicators are preferably LEDs, although any light, display, or other visual or audible indicator can instead be used. In the illustrated preferred embodiment for example, the portable operations device 14 is provided with two LEDs: one LED 86 for indicating the status of portable operations device operation and another LED 88 for indicating the status of the battery 70. In other embodiments of the present invention, the indicators 13 on the amplifier 12 described above can instead be located on the portable operations device 14 if desired. Preferably, the indicators 86, 88 on the portable operations device 14 are located in a position on the portable operations device 14 that is not covered or otherwise obstructed from user view when the portable operations device 14 is placed in a holster, is secured to or held within any other garment or wearable element as described above, or has the amplifier 12 and/or battery 70 mounted thereon as also described above. In the illustrated preferred embodiment for example, the indicators 86, 88 are located on the top of the portable operations device 14.

The indicators 86, 88 can provide any desired information to the user regarding the operation and connection of the portable operations device 14. Where an increased amount of information is desired, additional indicators can be added or the operation of the indicators can be changed. Where LED indicators are used, the LEDs can be conventional two-color LEDs that are also controlled to flash at different rates to indicate different portable operations device states. By way of example only, the portable operations device status LED 86 can be off when there is insufficient power to the portable operations device 14 or when power is disconnected thereto, flashing orange during initialization of the portable operations device 14, flashing alternating green and orange when no peripheral card 58 is detected in the bay 56, solid green when the portable operations device 14 is ready to receive physiological signals, flashing green when receiving such signals, and flashing orange when the storage peripheral memory card 58 is almost full. Also in this highly preferred embodiment, the battery status LED 88 can be off when the battery 70 is drained or is without sufficient power to operate the apparatus, can be solid red when the battery 70 is in such state while the portable operations device 14 is tethered to the host computer 16 (e.g., by cabling 40, 44, 46, 48), can be flashing green when the battery 70 is fully charged, and can be flashing red when battery power is low. One having ordinary skill in the art will appreciate that any combination of colors and flashing frequencies can be employed to provide the user with information regarding the state of the portable operations device 14 and the battery 70. It should also be noted that flashing or multi-colored LEDs can also be used for the amplifier LEDs 13 described above, if desired. In one preferred embodiment, the LED module 252 is implemented by the command interpreter 270. The microprocessor within the command interpreter 270 uses registers in the FPGA to drive the LED module 252.

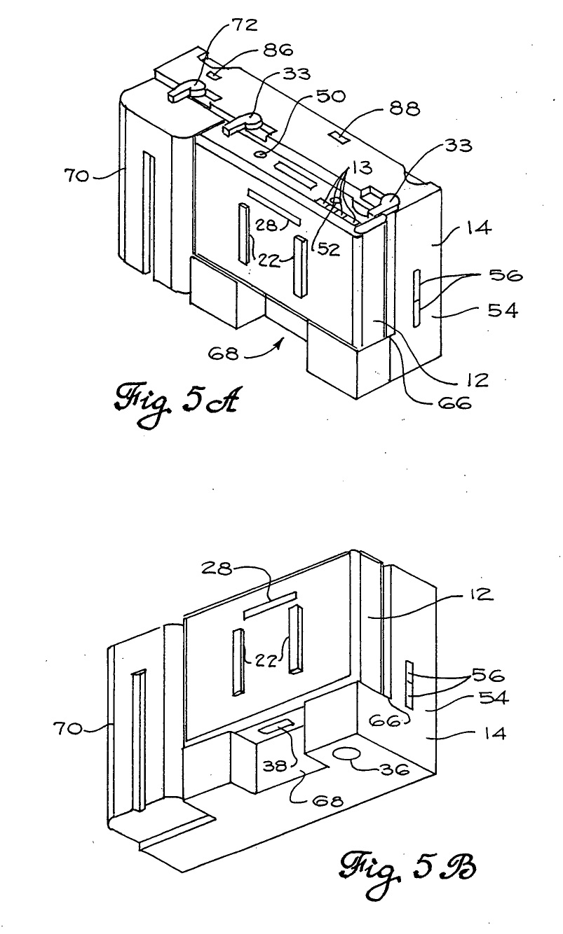

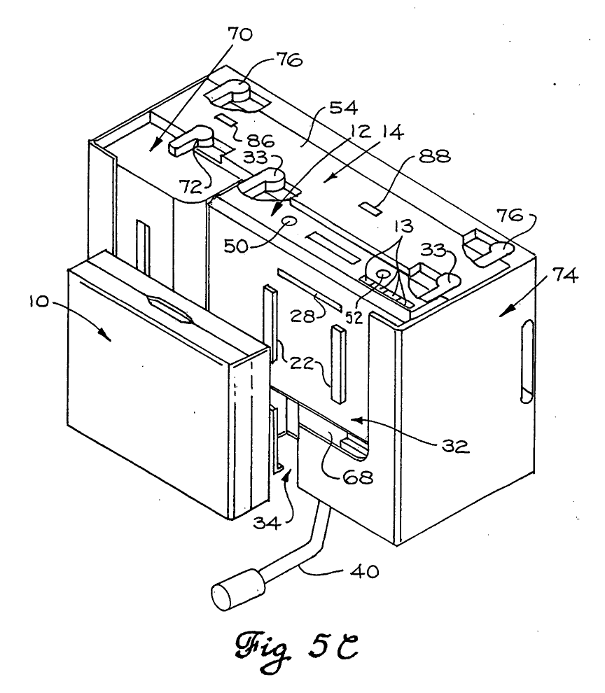

The portable operations device 14 can take any shape desired. However, with reference to FIGS. 5a and 5b, the portable operations device 14 is preferably shaped to define a seat 66 within or upon which the amplifier 12 is received. This seat 66 is preferably L-shaped as shown, but can surround more or fewer surfaces of the amplifier 12 in different embodiments. Preferably, the portable operations device 14 is shaped to receive the amplifier 12 already connected to the cable 40 extending therefrom. In the illustrated preferred embodiment for example, the bottom of the seat 66 preferably has a notch or recess 68 within which the end of the cable 40 is received when the amplifier 14 is installed in the portable operations device 14 and has the cable 40 connected thereto.

The amplifier 12 can be retained in the seat 66 in any of the manners described above with reference to the connection between the jackbox 10 and the amplifier 12, and most preferably is at least one latch 68 on the portable operations device housing 54 pivotable into and out of engagement with the amplifier 12. Alternatively, the latches 68 can be on the amplifier 12 for movement into and out of engagement with the portable operations device 14. Such latching mechanisms are conventional in nature and are not therefore described further herein.

Preferably, the portable operations device 14 is connected to a battery 70 for powering the portable operations device 14, amplifier 12, and the remainder of the monitoring system when not tethered to the host computer 16 (which preferably normally supplies these elements with power when tethered). Alternatively, the portable operations device 14, amplifer 12, and the remainder of the monitoring system could be powered from the mains power supply via a DC transformer. The battery 70 is preferably a conventional rechargeable battery and is releasably attached to the portable operations device 14 in any conventional manner. Most preferably, the battery 70 is releasably clipped into place on the portable operations device 14 by one or more conventional latches 72. The latches 72 can be the same or similar to those used in connecting the amplifier 12 to the portable operations device 14, if desired. In another preferred embodiment, the battery 70 is integral with or permanently connected to the portable operations device 14. In one highly preferred embodiment, the portable operations device 14 is capable of being powered by either the battery 70 for completely portable monitoring or a mains power supply for such uses as desktop monitoring.

The battery 70 or portable operations device 14 is preferably provided with a conventional battery threshold circuit capable of detecting when the battery 70 has reached a pre-set low power threshold and when the battery 70 has reached a pre-set insufficient power threshold. Preferably, both thresholds are monitored by the battery threshold circuit by monitoring battery voltage in any conventional manner. When the low power battery threshold has been reached during operation of the apparatus, this state is indicated by a battery status light 88 controlled by the battery threshold circuit (also in a conventional manner). If the insufficient power threshold is reached, the portable operations device preferably performs an orderly shutdown of the apparatus using the remaining battery power. Preferably, the portable operations device 14 is responsive to the battery circuit by not initializing signal transmission to the bay(s) 56 when the battery power detected is below the low power threshold. Battery threshold detection circuits are well known to those skilled in the art and are not therefore described in greater detail herein.

In addition to the battery 70, the portable operations device 14 can also be provided with an internal bridging battery 90 capable of temporarily operating the portable operations device 14 and amplifier 12 upon disconnection of the main battery 70. The bridging battery 90 is preferably a conventional rechargeable battery and permits the user to replace the main battery 70 without interrupting apparatus operation or needing to connect to another source of power while the main battery 70 is removed. Bridging batteries and their connection and operation are well known to those skilled in the art and are not therefore described further herein.

When the amplifier 12 is mounted upon the portable operations device 14, these two devices can be held to or secured upon the patient in any of the manners described above with reference to holding or securing the amplifier on the patient. Most preferably however, the amplifier 12, the portable operations device 14, and the battery 70 are received within a holster 74 worn on the patient (see FIG. 5c). This holster 74 is larger than amplifier holster 30 and is preferably used in place thereof. The holster 74 shape, manner of being worn upon the patient, manner of retaining the amplifier 12, portable operations device 14, and battery 70, and material (in addition to alternatives to these features) are similar to that described above with reference to the amplifier 12 in the amplifier holster 30. By employing a substantially open front connected to an opening in the holster bottom, the holster 74 provides easy access to and visual inspection of the amplifier 12 and battery 70, allows the jackbox 10 to be connected and disconnected from the amplifier 14 without removal from the holster 74, and permits insertion and removal of the amplifier 12, portable operations device 14, and battery 70 without disturbing an already connected cable 40. Also like the amplifier holster 30, the holster 74 most preferably permits connection and disconnection of the cable 40 without disturbing the devices in the holster 74, retains received devices via one or more latches 76, and is worn via belt apertures in the holster 74.

A significant advantage provided by the ability to mount the components of the present invention together as described above is the fact that the apparatus can be assembled as a self-contained integral unit, thereby making the apparatus easier to wear compared to conventional systems which typically employ multiple devices secured to or around the patient in a number of different locations. This also makes the apparatus worn by the patient easier to move and manipulate, and significantly decreases how much the equipment interferes with the patient in normal patient activities. The ability to mount the components as described also enables the apparatus to be placed upon and removed from the user much faster than conventional systems. While such advantages are significant, it should be noted that the portable operations device 14 can instead be held to or secured upon the patient separately from the amplifier 12 and battery 70, in which case each of these devices could be separately held to or secured upon the patient in any manner (such as those described above with reference to the jackbox 10). In such cases, the portable operations device 14 can be electrically connected to the amplifier 12 via cable or wireless transmission as also described above, and can be connected to the battery 70 or to a mains power supply by suitable electrical wiring.

In addition to receiving patient physiological signals from the amplifier 12, the portable operations device 14 can be provided with one or more sensors and one or more ports or jacks for connection to other patient monitoring equipment. By way of example only, the portable operations device 14 in the illustrated preferred embodiment has a microphone jack 78 for connection to a microphone, (although the portable operations device 14 could instead have a built-in microphone if desired), a light sensor 80 for detecting whether light is on or off during monitoring, at least one pneumatic port 82 (more preferably at least one pair of pneumatic ports) for connection to a conventional patient breathing monitoring device, a video input port (not shown) for conveying video images of the patient to the portable operations device 14, and one or more additional ports or jacks 84 for connection to such patient monitoring devices body position sensors, body temperature sensors, airflow transducers and/or strain gauges for breathing monitoring, blood pressure and pulse monitoring devices, and the like. A number of such devices are typically more commonly used for sleep and other patient monitoring rather than for EEG monitoring, but nevertheless can be included on the portable operations device 14 if desired. Any or all of the additional ports or jacks 84 can be high-level DC to AC inputs, such as high-level DC to 150 Hz AC inputs, as are well known to those skilled in the art. In one preferred embodiment, at least one of the additional inputs is a digital input. Although all of these input ports or jacks for connection to an event marker pendant and to a pulse oximeter are more preferably located on the amplifier 12 as described above, the portable operations device 14 can instead or in addition be provided with such ports or jacks.

A valuable feature of the present invention is the ability to add electrodes 18 to the apparatus for monitoring along additional channels while not sacrificing patient mobility and system simplicity. One manner in which more electrodes 18 can be added to the apparatus is by employing a jackbox 10 having a larger capacity and an amplifier 12 capable of receiving and amplifying the additional signals received from the jackbox 10. For example, a jackbox having 64 electrode connections and a larger amplifier used in conjunction therewith can be employed to result in a monitoring apparatus which is largely the same and operates in the same manner as the 32 electrode connector apparatus described above and illustrated in FIGS. 1 and 2 (with the exception of larger channel capacity). However, the present invention permits additional amplifiers and their corresponding jackboxes and electrodes to be quickly added to the monitoring apparatus without additional connections to the host computer, without interrupting system monitoring, and without loss of data. In one preferred embodiment, additional amplifiers can be added while the patient is disconnected from the host computer 16 and the system is under the control of the portable operations device 14. Specifically, in some highly preferred embodiments of the present invention, the amplifier 12 has an expansion communications jack or port 92 to which can be connected one or more additional amplifiers 12′ with electrodes 18′ connected thereto. In such embodiments, the present invention provides the ability to transmit physiological and communications signals between amplifiers and to thereby transmit system signals through as few as one cable (or wireless transmitter via the portable operations device 14 and receiver) to the host computer 16 or portable operations device 14. In contrast, the addition of an amplifier and its associated electrodes in conventional patient physiological monitoring systems requires an additional cable connection from the patient to the host computer, resulting in decreased patient freedom and mobility and increased patient discomfort.

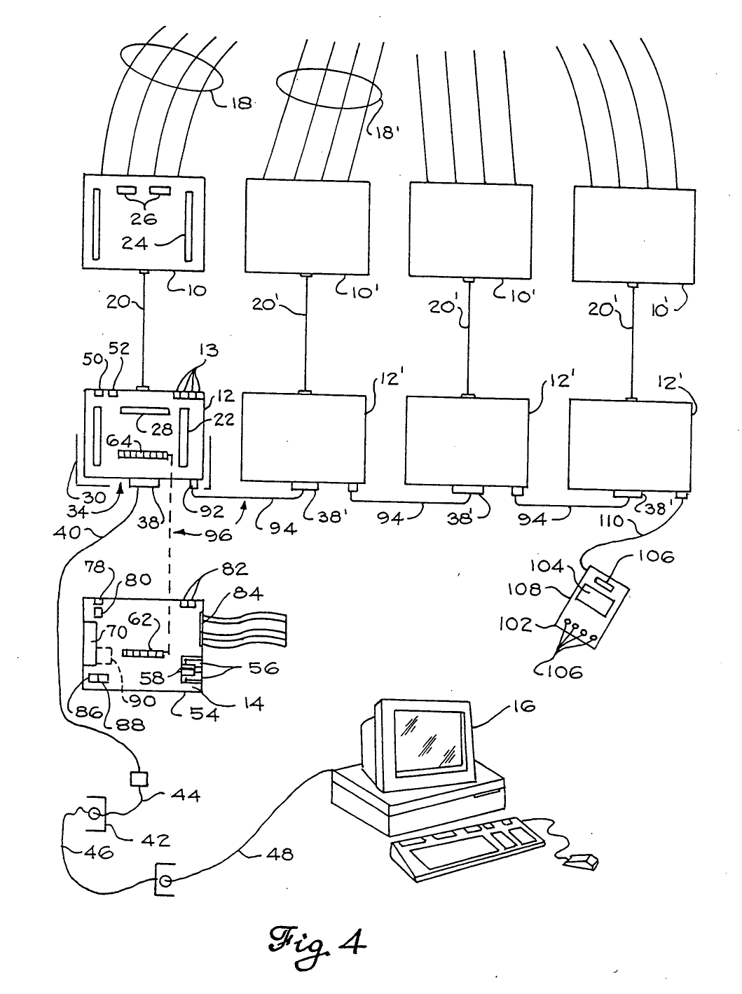

With reference to one preferred embodiment of the present invention illustrated in FIGS. 3 and 4, additional amplifiers 12′ are shown connected to the first amplifier 12. The additional amplifiers 12′ are each preferably connected to associated jackboxes 10′ and electrodes 18′ in the same manner as described above with reference to the embodiment of the present invention illustrated in FIGS. 1 and 2. In the illustrated preferred embodiment of FIGS. 3 and 4, three additional electrode, jackbox, and amplifier assemblies are connected to the first amplifier 12′. The additional electrodes, jackboxes, and amplifiers are preferably substantially the same and operate in substantially the same manner as the electrodes 18, jackbox 10, and amplifier 12 described above (with the exception of being connected to the amplifier 12 rather than directly to a portable operations device 14 or host computer 16). However, it should be noted that the additional electrode, jackbox, and amplifier assemblies can have different numbers and types of electrodes. Also, the manufacturer, type, and model of the additional jackboxes and amplifiers can be different from the jackbox 10 and amplifier 12 and can be different from each other. This modular feature of the present invention provides the user with the ability to add and remove amplifiers and jackboxes to the apparatus as needed for a particular application.

In the highly preferred embodiment shown in FIGS. 3 and 4, each jackbox 10, 10′ has 32 channels as described above with reference to the first jackbox 10. The resulting apparatus therefore has a 128 channel capacity, with preferably 32 channels of auxiliaries for the host interface card, and is presented by way of illustration only. For example, still larger channel capacities are possible with the substitution of larger jackboxes and amplifiers or with the addition of more electrode, jackbox, and amplifier assemblies.

The amplifiers 12′ added to the first amplifier 12 are preferably each connected in a daisy-chain configuration. Specifically, the amplifier jack or communications port 38′ of each amplifier 12′ is preferably connected by a cable 94 to the expansion communications port or jack 92 of another amplifier 12, 12′ in the apparatus. In other embodiments of the present invention, the amplifiers can be directly connected to one another without the use of cabling, such as in a manner similar to the communications connection between the portable operations device 14 and the first amplifier 12 described above. Such amplifier-to-amplifier connection can be side-by-side, above and below, face-to-face, or in any other manner desired. Although the portable operations device 14 illustrated in FIGS. 3 and 4 is shown connected to the first amplifier 12, it should be noted that in some preferred embodiments, the portable operations device 14 can be connected to any of the other amplifiers as desired (in which case the daisy chain of amplifiers 12, 12′ is preferably maintained with cables 94 connecting the communications ports 38, 38′ and expansion communications ports or jacks 92, 92′ of the amplifiers 12, 12′ as described above). Where the portable operations device 14 is connected at an opposite end of the daisy chain of amplifiers 12, 12′ (such as the last amplifier 12′ in FIGS. 3 and 4 rather than the first amplifier 12), the display device 102 can be connected through the aperture 36 of the portable operations device 14 to the amplifier 12′.

The expansion communications port or jack 92, 92′ and the communications port or jack 38, 38′ of each amplifier 12, 12′ are preferably internally connected to provide for signal transmission from one amplifier 12′ through another 12, 12′. The cables connecting the communications ports 38′ and the expansion communications ports 92, 92′ preferably have conventional releasable connector ends. When connected as described, the physiological signals from each additional amplifier 12′ are eventually transmitted to the first amplifier 12, while communications signals can be transmitted to the additional amplifiers 12′ via the first amplifier 12. The present invention employs a peripheral area network bus for physiological and communications signal control and routing and for supplying power to the amplifiers 12, 12′ and their connected jackboxes 10, 10′ from the main battery 70, bridging battery 90, and tethered power supply. Accordingly, the communications ports or jacks 38, 38′, the expansion communications ports or jacks 92, 92′, the amplifier circuitry connecting these ports or jacks 38, 38′, 92, 92′, in each amplifier 12, 12′, the cable(s) 94 connecting the amplifiers 12, 12′, the cable(s) 40, 44, 46, 48 connecting the first amplifier 12 to the host computer 16, and the communications connection 64, 62 connecting the first amplifier 12 to the portable operations device 14 represent at least part of a peripheral area network bus 96 of the apparatus.

Peripheral area network technology permits the addition and removal of peripheral devices to a peripheral area network bus without loss of data or communications between devices already on the peripheral area network bus (“hot plugging”). Generally speaking, peripheral devices can be added and removed to the end of the peripheral area network as desired. Accordingly, additional amplifiers 12′ and their associated jackboxes 10′ and electrodes 18′ can be quickly added or removed in the present invention without loss of patient physiological data.

To accomplish hot plugging, a signal from the FPGA of the command interpreter 206 is always looking for amplifiers that have been added, such as amplifier 12′, and begins downloading from the added amplifiers automatically. In one preferred embodiment, a signal is sent from the FPGA to the multiplexer to tell the multiplexer to send data to the A/D converter. The signal sent by the FPGA is timed for up to four amplifiers. If there is only one amplifier, one send signal is transmitted with three wait signals. If there are two amplifiers, two send signals are transmitted with two wait signals. If there are three amplifiers, three send signals are transmitted with one wait signal. If there are four amplifiers, four send signals are sent with no wait signals. For example, when amplifier 12′ is added to the daisy chain, the FPGA transmits a send signal to amplifier 12, a send signal to amplifier 12′, and then two wait signals before going back to amplifier 12. If a third amplifier were added, the FPGA would detect the addition of the amplifier and would begin downloading data from the third amplifier with the data from amplifiers 12 and 12′. The FPGA would then transmit a send signal to amplifier 12, a send signal to amplifier 12′, a send signal to the third amplifier, and one wait signal. Accordingly, the timing of the FPGA signals is such that the new data from the new amplifier can enter into the data stream with seamless synchronization with the other amplifiers.

The additional amplifiers 12′ and jackboxes 10′ can be worn by the patient in any of the manners described above with reference to the first amplifier 12 and jackbox 10. Preferably however, each additional amplifier 12′ is received within its own holster 30′ (not shown) which can be worn by the patient upon a belt or in any other manner desired. In other embodiments of the present invention, the additional holster(s) can be made larger to receive more than one amplifier 12′, if desired. It is even possible to employ a holster sufficiently large to receive all amplifiers 12, 12′ used in the apparatus. The jackboxes 10, 10′ in these alternative embodiments can be mounted directly upon their corresponding amplifiers 12, 12′ or can be connected thereto by cables 20, 20′ as also described above.

Where multiple jackboxes 10, 10′ are used in the present invention, it may be desirable to use the same reference electrode connected to one jackbox 10, 10′ as a reference electrode for one or more other jackboxes 10′, 10. In such a case, a jumper wire or cable can be connected to reference electrode connectors in the subject jackboxes 10, 10′ in a manner well known to those skilled in the art. The electrode connectors to which the desired reference electrode is connected can then be electrically connected to the jumper electrode connector. Typically, this connection is performed (under instruction from the user) by the system connected to the jackbox. Multiple jumpers can be connected between jackboxes 10, 10′ as desired.

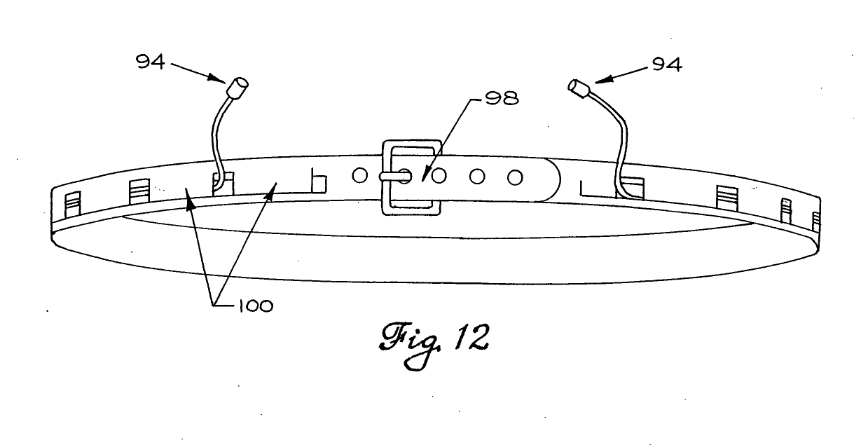

Especially where multiple amplifiers are used in the present invention (although applicable with even one amplifier 12), the belt 98 illustrated in FIG. 12 presents a convenient manner in which to arrange and wear the amplifiers 12, 12′ upon a patient. The belt 98 can be attached to each amplifier 12, 12′ in a number of manners well known to those skilled in the art, such as by a clip or hook on each amplifier 12, 12′, by hook and loop fastener material on the belt 98 and on each amplifier 12, 12′, by mating snaps or latches between the belt 98 and each amplifier 12, 12′, and the like. However, the belt 98 more preferably passes through belt apertures in each holster 30, 30′, 74 used in the apparatus of the present invention as described above.

After placing the amplifiers 12, 12′ (and portable operations device 14, if used) in desired locations on the belt 98, the amplifiers 12, 12′ can be connected by the cables 94 which are preferably received within flaps on the belt 98. Specifically, the belt 98 can have a series of flaps 100 attached thereto in any conventional manner, such as by being sewn, glued, riveted, or otherwise permanently fastened thereon, by hook and loop fastener material, by adhesive or cohesive tape, by one or more snaps, buttons, clips, pins, or other conventional releasable fasteners, by being integrally molded with the belt 98, and the like. These flaps 100 can be placed over the cables 94 in desired locations and can be fastened back upon the belt 98 in any of the manners just described. Preferably, at least one end of each flap 100 is releasable and re-attachable upon the belt 98 to permit cable adjustment, removal, and replacement. Apertures or notches between the flaps 100 along the belt permit the cables 94 to exit the belt 98 and to be connected as described above. Although flaps 100 are preferred to hold the cables 94 to the belt, a number of conventional fasteners can instead be used for this same purpose, including without limitation one or more clips, ties, lugs, clasps, and the like, each of which falls within the spirit and scope of the present invention.

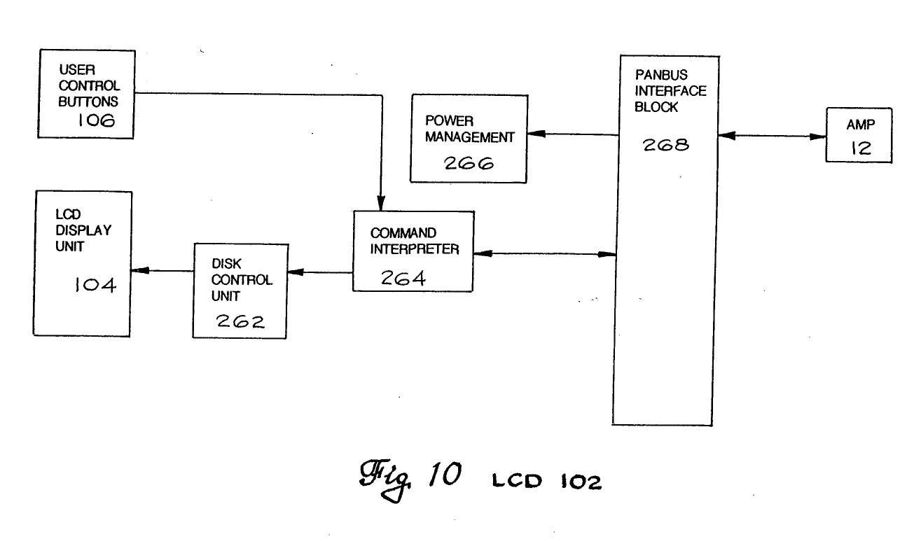

Some highly preferred embodiments of the present invention employ a handheld display unit 102 providing the user with at least some capability to monitor the patient physiological signals received by the apparatus. With reference to FIG. 10, the handheld display unit 102 is responsible for providing user input for system configuration and setup and feedback to the user when the user is not in physical proximity to the host computer. In one preferred embodiment, the handheld display unit 102 connects to the system at the last open daisy chain connector of the last amplifier via the peripheral area network bus cable, and only one handheld display unit 102 may be placed on any single peripheral area network bus. The handheld display unit 102 is mainly used during electrode application to monitor electrode impedance and to observe the quality of the signals from the electrodes to ensure that ambient noise is minimized. The handheld display unit 102 most preferably comprises six functional units although any number of units can be used satisfactorily. These units preferably comprise a display unit 104, a display control unit 262, a command interpreter 264, a power management unit 266, a peripheral area network bus interface 268, and user control buttons 106.

The display unit 104 displays the user information. In one preferred embodiment, the display screen is a conventional liquid crystal display (LCD) screen 104. The display screen can be luminescent or non-luminescent and can be color or mono-chromatic as desired. Preferably, the display unit 104 is capable of showing 44 electrode impedance designators per screen in 11 rows of four columns. The user can toggle between four screens to view a total of 128 channels of electrode impedance values and four bipolar channels of electrode impedance values. Preferably, the electrode impedance designators are updated no more often that once per 500 milliseconds and no less often than once per second.

The display control unit 262 drives the display unit 104 according to its input requirements and indicates the failure of any software. In one preferred embodiment, the display control unit 262 is an Epson. In an alternative embodiment, the display control functionality is placed in the FPGA of the command interpreter 270. The display control unit 262 provides the EEG data and any other patient data according to the input requirements of the display unit 104.

The command interpreter 264 receives and executes commands from the peripheral-area network bus. The command interpreter 264 configures the other units within and in addition to the handheld display unit 102. The command interpreter 264 saves configuration parameters, so that it can reinitialize the unit in case of power loss. The command interpreter 264 provides formatted data to the display control unit, including EEG data and any other patient data. Finally, the command interpreter 264 receives, formats, and provides data from the user control buttons 106 to the peripheral area network bus interface. In one preferred embodiment, the command interpreter 264 is implemented by a microprocessor.

The power management unit 266 generates all required power from the peripheral area network bus interface 268 and provides power-saving functionality to minimize the handheld display unit 102 power consumption. In one preferred embodiment, the command interpreter 264 oversees the power management unit 266. The command interpreter 264 controls the power to various portions of the circuit via a register in the FPGA.

The peripheral area network bus interface 268 receives data and commands from the peripheral area network bus and provides data and commands to the command interpreter 264, and in preferred embodiments, to other devices, such as an activation device or stimulator. The peripheral area network bus interface 268 formats and outputs responses and other information from the command interpreter 264 to the peripheral area network bus. The peripheral area network bus interface 268 receives, stores, and transmits the address of the handheld display unit 102 on the peripheral area network bus to the command interpreter 264 and generates an output address based on the input address. In one preferred embodiment, the peripheral area network bus interface 268 is implemented by the FPGA of command interpreter 264.

The user control buttons 106 provide user input to the command interpreter 264 to execute all required functionality. Preferably, the handheld display unit 102 includes at least one user control button 106 (such as buttons, levers, switches, and the like) permitting the user to control what is displayed on the display screen 104. The user-manipulable controls 106 are preferably conventional tactile membrane switch control keys under a Mylar® (DuPont Corporation) surface or other low-wear, waterproof, and durable surface. Other conventional key or button control types with or without an overlying surface can be used in alternate embodiments. In one preferred embodiment, the user control buttons 106 are read and processed by the command interpreter 264. The command interpreter 264 provides the user control button 106 data to the FPGA, which transmits the data to the host interface card 220 via the peripheral area network bus.

The handheld display unit 102 preferably has a waterproof housing 108 in which the display screen 104 and control keys 106 are located. Preferably, the handheld display unit 102 is connected to the rest of the apparatus by a conventional cable 110 over which physiological and apparatus communications signals can be transmitted and by which power can be supplied to the handheld display unit 102. The cable 110 is preferably connected to an available expansion communications jack or port 92 on one of the amplifiers 12, 12′. In less preferred embodiments, the cable 110 is connected to a port or jack on the portable operations device 14. Preferably, the cable 110 is releasably connectable to the communications jack or port 92, and can also be releasably connectable to a port or jack (not shown) on the handheld display unit 102. In other preferred embodiments of the present invention, the handheld display unit 102 can be provided with a conventional wireless receiver capable of receiving the physiological and apparatus communications signals from a wireless transmitter on an amplifier 12, 12′, on the portable operations device 14, or even on the host computer 16. More preferably, the handheld display unit 102 also has a wireless transmitter capable of transmitting command signals from the handheld display unit 102 to a wireless receiver on the amplifier 12, 12′, portable operations device 14, or host computer 16. Where the handheld display unit 102 is wireless, it is preferably provided with power from a conventional rechargeable or non-rechargeable on-board battery rather than via a power cord connection.

By connecting to an amplifier 12, 12′ of the apparatus as described above, the handheld display unit 102 is connected to the communications network of the apparatus (the peripheral area network bus of one preferred embodiment), and can receive, or preferably receive and control, the physiological signals transmitted from the amplifiers 12, 12′. Because the display unit 102 is handheld, the physiological signals being monitored by the apparatus can be viewed by a user without the need to view the host computer 16 and without sacrificing the patient mobility enabled by the present invention.

The controls 106 on the handheld display unit 102 can be manipulated by a user to view different physiological signals, physiological signal information, and apparatus information as desired. Preferably, the handheld display unit 102 is capable of displaying information in at least one of the following modes or displays: an electrode test (E-Test) mode, a calibration (CAL) mode, a pulse oximeter (SpO₂) display, and a Waveform display as will be described in more detail below.

Preferably, one or more controls 106 permit the user to “scroll” or otherwise move through different options on the display or “page” being shown on the display screen 104 and/or to move between pages shown on the display screen 104. For example, two of the keys 106 on the handheld display unit 102 are navigation keys in the form of up and down arrow keys used to perform this scrolling or moving function. Also preferably, another control 106 on the handheld display unit 102 permits a user to “select” or “enter” the choice or data highlighted by the scrolling or moving function just described. This ability to select the choice or data highlighted also preferably permits the user to navigate through multiple pages displayed on the display screen 104. In the illustrated preferred embodiment for example, one of keys 106 is a “Select” key which, when pressed, permits a user to select a highlighted entry on the display screen 104 and/or to navigate through different pages displayed thereon. Preferably, yet another control 106 on the handheld display unit 102 permits a user to automatically enter a main page in which the various modes or displays are presented from which the user can choose. For example, one of the keys 106 in the illustrated preferred embodiment is a “Mode” key which, when pressed, returns the user to a main page in which the four above-mentioned modes or displays are listed.

It should be noted that the various handheld display unit modes and displays mentioned above only represent preferred information to be displayed on the handheld display unit 102. In addition, the following description of each mode only represents a preferred manner of displaying such information and a preferred amount of such information. One having ordinary skill in the art will appreciate that the physiological and apparatus information being shown can be displayed in any number of different manners or formats and in greater or lesser detail as desired.

In the E-test mode, the display screen 104 preferably permits the user to select between “High” and “Full” displays. When in the Full mode, the handheld display unit 102 preferably displays a page in which a plurality of electrode threshold impedance values are displayed, any one of which can be selected to set the desired threshold impedance. Identification symbols or names for each electrode 18 are preferably received from the host computer 16 via the peripheral area network bus 96. When an impedance higher than the chosen threshold is detected by the apparatus (in a manner well known to those skilled in the art) such as when a poor electrode connection is made or when an electrode 18 is removed from the patient, this information can be transmitted to the host computer 16, to the handheld display unit 102, and more preferably to both the host computer 16 and the handheld display unit 102. By using the scroll and Select keys on the handheld display unit 102, the user can therefore set the desired threshold impedance values for each electrode 18 connected to the apparatus.

When in the High E-test mode, the handheld display unit 102 preferably displays at least one and more preferably simultaneously a plurality of electrode identifiers or names corresponding to those electrodes 18 which the system is monitoring. The measured impedance values can be displayed adjacent to each electrode identifier or name, and are measured in a manner well known to those skilled in the art). More preferably however, only one such impedance value is displayed at a time corresponding to the electrode identifier or name highlighted on the page. By pressing the navigation keys 106 on the handheld display unit 102, the user can scroll or move through the electrode identifiers or names to see the measured impedance value of each electrode. The impedance values displayed are preferably updated regularly to reflect changes in measured electrode impedance values. If necessary, multiple pages can be scrolled or otherwise navigated through by the user to see all information in both E-test mode types. In one preferred embodiment, the command interpreter 270 of the amplifier 12 includes an E-test signal generator.

In the CAL mode, at least one electrode trace representative of the settings in the amplifier analog signal conditioning section 202 is shown on the display screen 104. The electrode traces have a known voltage, amplitude, and period to allow for comparison with the signals from the patient electrodes 18. The user is then able to check the gain and filtering of the patient electrode 18 signals. In one highly preferred embodiment, four electrodes traces are displayed at once. By pressing the navigation keys 106 on the handheld display unit 102, the user can scroll or move though different electrode traces. In one preferred embodiment, the command interpreter 206 of the amplifier 12 includes a CAL signal generator. Additionally, the switching matrix within the electrode input block 200 of the amplifier 12 is responsible for the electronic patient disconnect during the CAL mode.

In the SpO₂ mode, the patient's SpO₂ and heart rate are preferably displayed in numerical format (although a graphical display of the patient's SpO₂ and an ECG display can instead or also be shown if desired). Like the electrode signals received and displayed in the E-test and CAL modes, the SpO₂ and heart rate information is transmitted to the handheld display unit 102 from the peripheral area network bus or other network connection to the amplifiers 12, 12′, and portable operations device 14 if used.

In the Waveform display mode, one electrode trace is preferably displayed with respect to a reference electrode 18. The electrode trace being displayed is preferably shown adjacent to the electrode identifier or name. By pressing the navigation keys 106 on the handheld display unit 102, the user can scroll or move though different electrode traces corresponding to the other electrodes 18 connected to the apparatus. The Waveform display is intended to be used during assembly setup to ascertain the quality of the data being received by the apparatus and to determine if unacceptable levels of power line noise or muscle artifact or other reducible artifacts are present in the signal being monitored.

In some highly preferred embodiments of the present invention such as the illustrated preferred embodiment, one of the user manipulable controls is a Deblock key 106 which is preferably functional during any mode of the handheld display unit 102. The Deblock key 106 can be depressed to perform a Deblock operation at any desired time. The analog signal conditioning section 202 of the amplifier 12 facilitates the Deblock function by shorting the blocking capacitors to ground. Deblocking operations are well known to those skilled in the art and are not therefore described further herein.

It will be appreciated that the handheld display unit 102 can be provided with any number and type of conventional controls 106 for the purpose of navigation, input selection, and other communication from the user to the handheld display unit. For example, the controls 106 can include dedicated keys for automatically entering each mode of the handheld display unit 102, a jog button to scroll through various available selections, and the like. As another example, the handheld display unit 102 can have a touch-sensitive screen by which user commands and inputs can be entered in addition to or in place of handheld display unit controls. Still other types of user control and input devices can be employed in the handheld display unit 102, each one of which falls within the spirit and scope of the present invention.

Operation of the present invention will now be described with reference to the above-described preferred embodiments of the present invention (although one having ordinary skill in the art will appreciate that the principles of operation as described apply equally to the alternative embodiments described above). In operation, a desired number of electrodes 18 are placed upon the patient in a manner well known to those skilled in the art, along with any other physiological sensors to be connected to the apparatus. The electrodes 18 are connected to as many jackboxes 10, 10′ as are needed before, during or after connection of the electrodes 18 and sensors to the patient. After the jackboxes 10, 10′ have been connected to their respective amplifiers 12, 12′, and the amplifier 12 has been tethered to the host computer 16 (if wireless transmission is not employed), patient monitoring can begin. Additional amplifiers 12′ can be connected to the first amplifier 12 as described above prior to initiation of patient monitoring.

In one preferred embodiment, once the desired number of amplifiers 12, 12′ and jackboxes 10, 10′ have been connected and tethered to the host computer, the host computer 16 begins patient monitoring by initiating a power on sequence for the amplifier 12. Preferably, when the amplifier 12 is tethered to the host computer 16, the amplifier 12 verifies that it is receiving a consistent response from the host interface card 220 during its default power up mode.

In another preferred embodiment, when the amplifier 12 is disconnected from the host computer 16, the amplifier 12 verifies that it is receiving a consistent response from the portable operations device 14, rather than from the host interface card 220, during its default power up mode. When the amplifier 12 is disconnected from the host computer 16, the amplifier 12 is initialized by a user manipulable control, such as a button, located on either the amplifier 12 or the portable operations device 14. In this manner, the amplifier 12 does not necessarily need to be tethered to the host computer 16 for the power on sequence. Accordingly, the amplifier 12 begins collecting data and the portable operations device 14 begins storing data without being initially tethered to the host computer 16. Preferably, the data stored on the portable operations device 14 is downloaded into the host interface card 220 when the amplifier 12 is reconnected to the host computer and is seamlessly synchronized with the new incoming patient data. Most preferably, the portable operations device 14 stores all of the patient data gathered in each of the monitoring sessions.

During patient monitoring while the apparatus is tethered to the host computer 16, physiological signals from the electrodes 18 are transmitted to the amplifiers 12, 12′ via the jackboxes 10, 10′ and then to the host computer 16 by the cable(s) 40, 44, 46, 48. Additional physiological signals from the monitoring devices connected to the amplifier 12 are also preferably transmitted to the host computer 16 along the cables 40, 44, 46, 48.

When the patient is tethered to the host computer 16, the patient electrode data is ultimately transmitted to the host interface card 220. When the patient is tethered, the host interface card 220 acts as the bus master. The electrode input block 200 of the amplifier 12 receives the patient electrode signals from the patient electrodes 18. The patient electrode signals are then sent to the analog signal conditioning section 202 for amplification and filtration. The various channels of amplified and filtered patient electrode signals are sent to the A/D converter 204 via a multiplexer. The A/D converter 204 digitizes the patient electrode signals and sends the data through the peripheral area network bus to the FPGA of the command interpreter 270. From the portable operations device 14, the data is transmitted over the peripheral area network bus to the host interface card 220. In one preferred embodiment, video and audio data is transmitted over the peripheral area network bus to the host interface card 220. The data is filtered by the filtering module 228 of the host interface card 220 and sent to the global storage area 230.

At any time during patient monitoring, additional amplifiers 12′ can be added or removed from the apparatus by connecting or disconnecting the additional amplifiers 12′ by the cable 94 connecting the additional amplifiers 12′ to the next amplifier 12, 12′ in the daisy chain. By virtue of one preferred peripheral area network bus used to connect the amplifiers 12, 12′ of the present invention, additional amplifiers 12′ can be seamlessly added and removed from the apparatus and can be initialized automatically upon being added without the loss or corruption of data from the other devices connected to the peripheral area network bus (such as data passing from the first jackbox 10 through the first amplifier 12 and to the host computer 16). Similarly, the handheld display unit 102 can be connected to the apparatus as described above even during patient monitoring.

When disconnection of the patient from the host computer 16 is desired, the amplifier 12 is preferably inserted within and electrically connected to the portable operations device 14 (if not already done). This connection to the portable operations device 14 can be made without disturbing operation of the apparatus because the cable 40 extending from the amplifier 12 need not be disconnected to insert the amplifier 12 into the portable operations device 14. The apparatus is preferably disconnected from the host computer 16 by disconnecting the amplifier tether cable 40 from the extension cable 44 leading to the wall jack or port 42. Where removable peripheral cards 58 in the portable operations device 14 are employed to receive data from the amplifier 12 rather than non-removable memory media in the portable operations device 14, the user first inserts one or more such peripheral cards 58 into the bay(s) 56 of the portable operations device 14. Upon disconnection, power is supplied to the apparatus via the battery 70 on the portable operations device 14.

When the host interface card 220 is the bus master, the portable operations device 14, as the bus slave, monitors the peripheral area network bus for data from the amplifier 12 and stores the data in RAM, but not on the PCMCIA interface 240. The portable operations device 14 waits for the disconnection of the tether, at which time it becomes the bus master.

In another preferred embodiment, the portable operations device 14 does not monitor the peripheral area network bus to detect when the host computer 16 is disconnected from the amplifier 12. Rather, the portable operations device 14 begins collecting and storing data in response to a manual user manipulation, such as by pushing a button on the portable operations device 16 or the amplifier 12 coupled to the command interpreter 270 or by entering a command on the host computer 16. Once the portable operations device 14 is manually activated for data storage, the host computer 16 ceases collecting data in response to another manual user manipulation and the amplifier 12 is untethered from the host computer 16. In the above-described preferred embodiment, the portable operations device 14 is operable without a microprocessor as the command interpreter 270.

When the patient is disconnected from the host computer 16, the patient electrode data is ultimately transmitted to the host interface card 220, but the data is stored on the portable operations device 14 until the patient is again tethered to the host computer 16. In one preferred embodiment, compressed video and audio data is stored on the portable operations device 14. When the portable operations device 14 detects the loss of communication with the host computer 16, the portable operations device 14 becomes the bus master. The electrode input block 200 receives the patient electrode signals from the patient electrodes 18. The patient electrode signals are then sent to the analog signal conditioning section 202 for amplification and filtration. The various channels of amplified and filtered patient electrode signals are sent to the A/D converter 204 via a multiplexer. The A/D converter 204 digitizes the patient electrode signals and sends the data through the peripheral area network bus to the FPGA of the command interpreter 270. The data is filtered by the portable operations device filtering unit 244 and compressed by the compression unit 246. The compressed data is then sent to the PCMCIA interface 240 for storage. The re-routing of physiological signals from the amplifier port or jack 38 to the bay 56 and peripheral card 58 of PCMCIA interface 240 upon cable disconnection is seamless and results in no loss of data.

While the amplifier 12 remains untethered to the host computer 16, the physiological signals are sent to the PCMCIA interface 240 and preferably either stored upon the peripheral card 58 in the case of a memory card or are transmitted from the peripheral card 58 to a receiver on the host computer 16 in the case of a transmitter card. The signals continue to be transmitted to the bay 56 until the peripheral card 58 is full in the case of a memory card, until the battery 70 has insufficient power to operate the apparatus, or until the cable 40 is reconnected to the amplifier 12.

When the patient is disconnected from the host computer 16, other physiological signals from the amplifier 12 and from the additional amplifiers 12′ connected thereto via the expansion communications jack or port 92 (including signals from the electrodes 18, 18′ and signals from any other physiological monitoring device connected to the amplifiers 12, 12′ such as an event marker pendant or an SpO₂ monitor connected to amplifier ports or jacks 52, 50) are then routed to the portable operations device 14 and to the bay 56 and peripheral card 58 therein rather than to the amplifier port or jack 38.

When the portable operations device 14 is the bus master, it issues all commands and receives all data. The portable operations device 14 also sends a signal to the host interface card 220 in an attempt to determine when the host interface card 220 is reconnected. This signal includes the current patient's identification information. When the host interface card 220 is detected, the FPGA of command interpreter 270 provides an interrupt to the microprocessor of command interpreter 270 to indicate that the host interface card 220 is now back online and ready to become the bus master. The command interpreter 270 then sends a signal to switch the portable operations device 14 from master to slave mode. In one preferred embodiment, the command interpreter 270 controls the switching between master and slave on the peripheral area network bus. However, the host interface card command interpreter 264 could also control the switching between master and slave.

In another preferred embodiment, the portable operations device 14 does not send a signal to the host interface card 220 in an attempt to determine when the host interface card is reconnected. Rather, the amplifier 12 is tethered to the host computer 16, and host interface card 220 begins collecting data in response to manual user manipulation, such as by pushing a button on the portable operations device 16 or the amplifier 12 coupled to the command interpreter 270 or by entering a command on the host computer 16 as described above. Once the host interface card 220 is manually activated for data collection, the portable operations device 14 ceases collecting data in response to another manual user manipulation. In the above-described preferred embodiment, the portable operations device 14 is operable without a microprocessor as the command interpreter 270.

Once the patient is again tethered to the host computer 16, the data stored on the PCMCIA interface 240 is transmitted through the peripheral area network bus to the host interface card 220, while the host interface card 220 continues to receive new patient data from the amplifier 12. During the download of the old patient data from the PCMCIA interface 240, the old patient data is seamlessly interjected into the global storage area 230 ahead of the new patient data that is being acquired. The patient can be tethered and untethered to the host computer 16 at any time without any loss of patient data due to the ability to simultaneously acquire new data and repatriate the new data with the old data. Following reconnection of the amplifier 12 to the host computer 16 via the amplifier cable 40, the physiological signals from the amplifier 12 and from the additional amplifiers 12′ (including the electrode signals 18, 18′ and signals from any other physiological monitoring device connected to the amplifiers 12, 12′) are re-routed to the amplifier port or jack 38 and to the host computer 16 rather than to the portable operations device 14. Also following this reconnection, the physiological data stored in the peripheral card 58 (in the case of a memory card) is preferably transmitted from the peripheral card 58 to the amplifier 12 and to the host computer 16 via the amplifier jack or port 38 and cable 40.

Alternatively, the user can remove the peripheral memory card 58 with the physiological signal data thereon, can insert the peripheral memory card 58 into the host computer 16 in a conventional manner, and can download the data to the host computer 16 to be repatriated with the physiological signals transmitted via cable 40, 44, 46, 48 if desired.

Where the peripheral card 58 in the bay 56 is a transmitter card, disconnection of the amplifier 12 from the host computer 16 preferably causes the physiological signals received by the portable operations device 14 to be transmitted by the peripheral card 58 to a receiver on or connected to the host computer 16. In this manner, physiological signal data is not lost upon disconnection of the apparatus from the host computer 16. In more preferred embodiments of the present invention, a transmitter peripheral card 58 in the bay 56 functions in this manner until communication with the receiver on or connected to the host computer 16 is lost, at which time physiological data is instead recorded upon a memory peripheral card 58 also in the bay 56 until communication is re-established with the receiver or the amplifier 12 is reconnected to the host computer 16. Following re-establishment of communication with the receiver, physiological data can once again be transmitted via the transmitter peripheral card 58 to the host computer 16, along with the data stored on the memory peripheral card 58, if desired. Alternatively, the data stored on the memory peripheral card 58 can be repatriated with the transmitted data after reconnection of the amplifier 12 with the host computer 16 or by removing the memory peripheral card 58 from the portable operations device 14 and installing the memory peripheral card 58 into the host computer as described above. The data stored on the memory peripheral card 58 is repatriated with the transmitted data simultaneously with the acquisition of the new data. Systems and devices for detecting the loss and acquisition of communication between wireless devices are well known to those skilled in the art and are not therefore described further herein.

Because the portable operations device 14 is preferably capable of receiving multiple peripheral cards 58, it is possible to provide for a virtually endless amount of data to be saved to memory peripheral cards 58 if desired. Specifically, physiological signals can be transmitted to and saved upon one of the memory peripheral cards 58 in the bay 56 while a full memory peripheral card 58 is being removed and replaced by another memory peripheral card 58. Routing of physiological data between peripheral cards (e.g., routing data from a first peripheral card to a second peripheral card when a full memory is detected in the first peripheral card) is preferably performed by the command interpreter 270 on the portable operations device 14 in a manner well known to those skilled in the art. Alternatively, the portable operations device 14 can be provided with a buffer memory capable of temporarily storing data while a full memory peripheral card 58 is exchanged for a new memory peripheral card 58. The buffer memory can automatically repatriate data saved therein to the new memory peripheral card 58 after its installation in the bay 56. Such buffer memories, their connection, and operation are also well known to those skilled in the art and are not therefore described further herein.

It may be desirable to recharge the battery 70 prior to reconnecting the amplifier 14 with the host computer 16. This can occur, for example, where the patient is untethered from the host computer 16 for extended periods of time during which physiological data is transmitted by a transmitter peripheral card 58 in the portable operations device bay 56, where the rate of memory consumption of a memory peripheral card 58 in the bay 56 exceeds the rate at which battery power is depleted, where the patient is not near the host computer 16, or when the battery 70 approaches low power for any other reason. For this purpose, the battery 70 or the portable operations device can be connected to a conventional battery charger having a power cord that can be connected to a source of power as needed.

Although operation of the apparatus as described above is with reference to an apparatus originally tethered to the host computer 16 at the beginning of physiological signal monitoring, it should be noted that the present invention preferably need not be tethered to a host computer to begin such monitoring. The portable operations device 14 is preferably fully capable of apparatus initialization and startup processes without assistance from the host computer 16. This capability is particularly valuable in many applications, such as for apparatus use by emergency medical technicians not at a medical care facility, for connection to and monitoring of a patient that cannot yet be moved to a location near the host computer 16, etc. It should also be noted that during a patient's monitoring session, the apparatus of the present invention need not necessarily be disconnected from and reconnected to the same host computer 16. The apparatus can be tethered to different host computers during the same session as necessary or convenient, such as where the patient is moved to different areas of a medical facility or even between medical facilities. Physiological signal data from the patients' monitoring session can be collected or repatriated to the same host computer 16 at a later time, if desired.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

For example, the preferred embodiments described above are each directed to electroencephalography (EEG) monitoring and employ conventional electrodes suitable for this type of monitoring. However, the principles of the present invention are equally applicable to monitoring virtually any other physiological activity of a patient, and result in the same or similar benefits. The present invention can be used in electrocorticography (ECₒG) for monitoring electrodes placed on the surface of the brain, electromyography (EMG) for monitoring and potentially stimulating muscle activity, electrocardiology (ECG) for monitoring heart activity, electrooculography (EOG) for monitoring ocular activity, polysomnography (PSG) for sleep monitoring, magnetoencephalography (MEG) for monitoring magnetic bioelectric signals from the brain, maternal/fetal monitoring, respiratory monitoring, various types of ambulatory monitoring, general data acquisition, or virtually any other type of patient monitoring.

In this regard, the electrodes 18 employed to receive the physiological signals can be different and adapted to each particular monitoring application as is well known to those skilled in the art. Fewer (as few as one) or greater numbers of electrodes being of all the same type or different types can therefore be used in different monitoring applications, and can be located on any portion on or within a patient's body depending primarily upon the physiological phenomenon being monitored. The present invention is not limited to electrophysiology monitoring, and can instead be employed to monitor any physiological aspect of a patient, whether related to electrical signals of the patient or not. In order to configure the present invention to other types of monitoring, different algorithms for each type of monitoring are created and downloaded into the command interpreters 206, 224, and 270 in order to implement the specific requirements for each type of monitoring.

If desired, combinations of electrophysiology sensors and other sensor types can be connected to the same apparatus. Any or all of the sensors may not require the use of an amplifier 12, 12′, in which case remaining necessary signal processing can be performed by the portable operations device 14 and/or at the host computer 16. In short, although EEG signals are monitored in one preferred embodiments described above, the present invention is not limited to such monitoring and can be used with the same, similar, or different types of sensors on or in any area of the patient's body for the purpose of monitoring any physiological activity of a patient.

In addition, although the jackboxes 10, 10′, amplifiers 12, 12′, portable operations device 14, and battery 70 are described above and illustrated in the figures as being separate elements each having its own housing, it should be noted that any one or more of these components can be combined within one housing as a single integral unit in which the circuitry of these components are either maintained separate or are combined to any degree desired. For example, a jackbox 10 can be combined with an amplifier 12 as a single integral unit. As another example, the handheld display unit 102 can be combined with the portable operations device 14 or an amplifier 12 as a single integral unit.

In one preferred embodiment, the present invention is used for sleep monitoring, such as for the control and monitoring of continuous positive airway pressure (CPAP), bilevel positive airway pressure (BiPAP), or a variable positive airway pressure. Preferably, sleep monitoring also includes such monitoring devices as pressure transducers, strain guages, pulse oximeters, limb electrodes, pneumatic ports, and microphones. Most preferably, the present invention controls the level of positive airway pressure administered to the patient in response to the analysis of the signals from each of the above-mentioned monitoring devices. In controlling the level of positive airway pressure that is administered, the present invention is able to increase or decrease the level administered depending on which stage of sleep the patient is currently in as determined from the EEG data and the other patient data.

In one preferred embodiment, the present invention is used for respiratory monitoring, with monitoring devices such as respiratory transducers, thermisters, pressure transducers, piezoelectric devices, and vibration or sound sensors.

In one preferred embodiment, the present invention is used for ambulatory devices, such as home monitoring units for EEG, ECG, or any other patient data, electrophysiological or otherwise.

In one preferred embodiment, the present invention is used for EMG monitoring for purposes such as specific types of sleep monitoring or for back injury assessments. In sleep monitoring, EMG data is used for disorders such as restless leg syndrome or REM sleep behavior disorder. For restless leg syndrome, EMG electrodes are attached to the legs to determine the level of muscle activity in the legs and to correlate that activity with the EEG data. For REM sleep behavior disorder, EMG electrodes are attached to the skin around the eyes to determine the muscle activity of the eyes and to correlate that activity with the EEG data. For back injury assessments, it is often difficult to determine the level of pain patients with back injury are in. By monitoring the EMG activity of the back muscles, clinicians can determine the level of back injury more accurately and quantitatively.

In order to monitor EMG signals, changes must be made to the EEG monitoring configuration or a completely different algorithm must be downloaded into the command interpreters. Specifically, the sample rate for the digital signal processor must be changed via reconfiguration of the FPGA. Generally, EMG signals are at a frequency of 3000 Hz to 10 kHz, while EEG signals are only at a frequency of 0.5 Hz to 100 Hz. This difference in frequency results in the need for different sample rates for the digital signal processor for the EMG and EEG signals. Accordingly, less channels of EMG signals are acquired but at a higher sample rate. By way of example only, 16 channels of EMG signals are acquired at a sample rate of 4000 samples per second, rather than 32 channels of EEG signals being acquired at a sample rate of 2000 samples per second.

In one highly preferred embodiment, the present invention can monitor both EEG signals and EMG signals simultaneously. One way of accomplishing this task is to use two amplifiers with different sample rates provided by the FPGA within each amplifier. Another way of accomplishing this task is to sample the EEG signals at the same higher rate that the EMG signals are sampled at, and to later disregard the unnecessary EEG signal samples.

In one preferred embodiment, the present invention is used in cardiac electrophysiology studies. Generally, fewer channels are needed for ECG studies than in EEG studies. However, in one highly preferred embodiment for use in cardiac electrophysiology, a 128 electrode sock-like device encapsulates the heart in order to study its functioning.

In one preferred embodiment, the present invention is adapted for use in veterinary medicine for the monitoring and control of biopotential signals in animals.

As used herein and in the appended claims, when one element or device is said to be “coupled” to another, this does not necessarily mean that one element is fastened, secured, or otherwise directly attached to another element. Instead, the term “coupled” means that one element is either connected directly or indirectly to another element or is in mechanical or electrical communication with another element. Also as used herein and in the appended claims, the terms “input” and “output” refer to any electronic or communications connector of any shape and type, whether male, female, or otherwise, and need not necessarily be a releasable connector. In this regard, inputs and outputs of a device are those elements by which signals or data are received into or sent from the device, respectively. In some cases, the same element or elements of a device can be both an input and an output.

1. An electroencephalographic patient monitoring system for monitoring electrical physiological signals from the brain of a patient and for communication with a host computer, the electroencephalographic patent monitoring system comprising:
a host computer interface;
an amplifier having a communications port for transmitting electroencephalographic signals to the host computer; and
a portable operations device coupled to the amplifier, the portable operations device having
a memory; and
a controller responsive to disconnection of communication between the communications port and the host computer by transmitting electroencephalographic signals to the memory.

2. The electroencephalographic patient monitoring system as claimed in claim 1, further comprising a bay in the portable operations device within which the memory is removably received.

3. The electroencephalographic patient monitoring system as claimed in claim 2, wherein the memory is on a peripheral card removably received within the bay in the portable operations device.

4. The electroencephalographic patient monitoring system as claimed in claim 1, further comprising a cable for connecting the communications port with the host computer interface, wherein electroencephalographic signals are transmitted from the amplifier to the host computer interface via the cable.

5. The electroencephalographic patient monitoring system as claimed in claim 4, wherein power is supplied to the amplifier and to the portable operations device via the cable.

6. The electroencephalographic patient monitoring system as claimed in claim 5, further comprising a battery coupled to the portable operations device, the amplifier and the portable operations device powered by the battery when the cable between the amplifier and the host computer is disconnected.

7. The electroencephalographic patient monitoring system as claimed in claim 1, further comprising:
a wireless transmitter coupled to the communications port; and
a wireless receiver for coupling to the host computer interface,
wherein communication between the communications port and the host computer interface is via the wireless transmitter and receiver.

8. The electroencephalographic patient monitoring system as claimed in claim 7, further comprising a battery coupled to and powering the portable operations device and amplifier.

9. The electroencephalographic patient monitoring system as claimed in claim 1, wherein the controller is responsive to re-establishment of communication between the communications port and the host computer by routing new electroencephalographic signals from the patient to the host computer and by transmitting electroencephalographic signals in the memory to the host computer.

10. The electroencephalographic patient monitoring system as claimed in claim 1, wherein the controller is responsive to re-establishment of communication between the communications port and the host computer by simultaneously routing new electroencephalographic signals from the patient to the host computer and transmitting electroencephalographic signals in the memory to the host computer.

11. The electroencephalographic patient monitoring system as claimed in claim 1, further comprising:
at least one electrode operable to receive electroencephalographic signals from the patient's brain; and
a jackbox coupled to the amplifier and to the at least one electrode to transmit electroencephalographic signals from the patient's brain to the amplifier.

12. The electroencephalographic patient monitoring system as claimed in claim 11, wherein the portable operations device has
an input coupled to the controller and operable to receive the electroencephalographic signals from the amplifier; and
at least one other input coupled to the controller and operable to receive at least one other type of physiological signal from the patient.

13. The electroencephalographic patient monitoring system as claimed in claim 12, wherein the at least one other input is a microphone jack.

14. The electroencephalographic patient monitoring system as claimed in claim 12, wherein the at least one other input is a light sensor.

15. The electroencephalographic patient monitoring system as claimed in claim 12, wherein the at least one other input is a jack releasably connectable to a patient physiological signal monitoring device.

16. The electroencephalographic patient monitoring system as claimed in claim 1, wherein the amplifier has an expansion port to which at least one additional amplifier can be releasably coupled.

17. The electroencephalographic patient monitoring system as claimed in claim 16, further comprising a peripheral area network bus defined in part by the expansion port of the amplifier.

18. The electroencephalographic patient monitoring system as claimed in claim 1, wherein the amplifier and the portable operations device are wearable upon the patient.

19. The electroencephalographic patient monitoring system as claimed in claim 1, further comprising a communications port for transmitting video signals to the host computer.

20. The electroencephalographic patient monitoring system as claimed in claim 1, further comprising a communications port for transmitting audio signals to the host computer.

21. The electroencephalographic patient monitoring system as claimed in claim 1, further comprising a stimulation generator in the amplifier for cortical stimulation.

22. A method of monitoring electroencephalographic signals from a patient's brain, comprising:
amplifying electroencephalographic signals from the patient's brain;
transmitting amplified electroencephalographic signals to a host computer;
detecting loss of communication with the host computer;
transmitting amplified electroencephalographic signals to a portable memory device on the patient in response to detecting loss of communication with the host computer;
storing the amplified electroencephalographic signals as data in the portable memory device while communication with the host computer is lost;
detecting re-establishment of communication with the host computer; and
transmitting new amplified electroencephalographic signals to the host computer.

23. The method as claimed in claim 22, wherein the portable memory device is a peripheral card coupled to the amplifier.

24. The method as claimed in claim 23, wherein the portable memory device is coupled to the amplifier via a bay on the patient, the method further comprising removing the peripheral card from the bay.

25. The method as claimed in claim 22, further comprising removing the peripheral card from the patient.

26. The method as claimed in claim 22, further comprising transmitting data from the portable memory device after detecting re-establishment of communication with the host computer.

27. The method as claimed in claim 22, further comprising transmitting data from the portable memory device simultaneously with transmitting the new amplified electroencephalographic signals to the host computer.

28. The method as claimed in claim 22, wherein electroencephalographic signals from the patient's brain are amplified by an amplifier worn by the patient.

29. The method as claimed in claim 28, wherein amplified electroencephalographic signals are transmitted to the host computer via a cable coupled to the amplifier and to the host computer, the method further comprising disconnecting the cable.

30. The method as claimed in claim 29, further comprising re-connecting the cable prior to detecting re-establishment of communication with the host computer.

31. The method as claimed in claim 22, wherein the electroencephalographic signals are amplified by at least two amplifiers coupled together.

32. The method as claimed in claim 31, wherein the at least two amplifiers are coupled together along a peripheral area network bus.

33. The method as claimed in claim 31, further comprising:
coupling a first amplifier of the at least two amplifiers to the host computer; and
coupling a second amplifier of the at least two amplifiers to the first amplifier, wherein
transmitting electroencephalographic signals includes transmitting a first plurality of electroencephalographic signals from the second amplifier to the first amplifier and transmitting a second plurality of electroencephalographic signals with the first plurality of electroencephalographic signals from the first amplifier to the host computer.

34. The method as claimed in claim 22, further comprising transmitting video signals to the host computer.

35. The method claimed in claim 22, further comprising transmitting audio signals to the host computer.

36. The method as claimed in claim 22, further comprising transmitting stimulation signals to the patient.

37. The method as claimed in claim 22, further comprising:
coupling an amplifier to the portable memory device via a first input prior to amplifying electroencephalographic signals from the patient's brain;
coupling at least one physiological signal monitoring device to the memory via at least one other input; and
transmitting physiological signals of the patient to the memory from the at least one physiological signal monitoring device.

38. The method as claimed in claim 22, further comprising:
coupling at least one electrode to the patient's brain;
coupling the at least one electrode to a jackbox worn by the patient; and
coupling the jackbox to the amplifier prior to amplifying electroencephalographic signals from the patient's brain.

39. A portable electroencephalographic operations device for receiving and directing patient electroencephalographic signals in a system having an amplifier and a host computer, the portable electroencephalographic operations device comprising:
an input adapted to receive electroencephalographic signals from the amplifier;
an output adapted to transmit electroencephalographic signals to the host computer;
a memory in communication with the input and output; and
a controller coupled to the input, output and memory and operable to transmit electroencephalographic signals to the memory responsive to loss of communication between the amplifier and the host computer and to transmit electroencephalographic signals stored in the memory to the host computer upon re-establishment of communication between the amplifier and the host computer.

40. The portable electroencephalographic operations device as claimed in claim 39, wherein the output transmits electroencephalographic signals to the host computer via the amplifier.

41. The portable electroencephalographic operations device as claimed in claim 40, further comprising a communications port adapted to connect to the amplifier, wherein the input and output are defined by the communications port.

42. The portable electroencephalographic operations device as claimed in claim 39, wherein the output is a wireless transmitter operable to transmit electroencephalographic signals wirelessly to the host computer.

43. The portable electroencephalographic operations device as claimed in claim 39, further comprising a housing within which the input, output, memory, and controller are at least partially enclosed, wherein the housing is adapted to be worn upon a patient.

44. The portable electroencephalographic operations device as claimed in claim 43, further comprising a belt to which the housing is coupled.

45. The portable electroencephalographic operations device as claimed in claim 39, wherein the memory is at least one removable peripheral card.

46. The portable electroencephalographic operations device as claimed in claim 39, further comprising at least one bay coupled to the controller and to the input and output, the at least one bay adapted to removably receive a peripheral card.

47. The portable electroencephalographic operations device as claimed in claim 46, wherein the memory is a peripheral card removably received within the at least one bay.

48. The portable electroencephalographic operations device as claimed in claim 39, wherein the portable electroencephalographic operations device is shaped to removably receive at least part of the amplifier.

49. The portable electroencephalographic operations device as claimed in claim 48, further comprising a communications port adapted to connect to the amplifier upon insertion of the amplifier in the portable electroencephalographic operations device.

50. The portable electroencephalographic operations device as claimed in claim 48 for use in conjunction with an amplifier connected by a cable to the host computer, wherein the portable electroencephalographic operations device has an open portion shaped to receive the cable connected to the amplifier.

51. The portable electroencephalographic operations device as claimed in claim 39 for use in conjunction with a battery and an amplifier releasably connected by a cable to the host computer, the portable electroencephalographic operations device having:
a first power mode in which the cable is disconnected and the portable electroencephalographic operations device is powered by the battery; and
a second power mode in which the cable is connected and the portable electroencephalographic operations device is powered via the cable.

52. The portable electroencephalographic operations device as claimed in claim 39, further comprising at least one additional input coupled to the controller, releasably connectable to a physiological signal monitoring device, and adapted to receive physiological signals from the physiological signal monitoring device.

53. A method of controlling patient electroencephalographic signal transmission, comprising:
transmitting patient electroencephalographic signals through an amplifier to a host computer;
coupling a portable operations device to the amplifier;
disconnecting the amplifier from the host computer;
transferring transmission of patient electroencephalographic signals from the amplifier to the portable operations device;
reconnecting the amplifier to the host computer; and
transferring transmission of patient electroencephalographic signals from the amplifier to the host computer after reconnection of the amplifier to the host computer.

54. The method as claimed in claim 53, wherein transferring transmission of patient electroencephalographic signals from the amplifier to the portable operations device is performed responsive to disconnection of the amplifier from the host computer.

55. The method as claimed in claim 53, wherein transferring transmission of patient electroencephalographic signals from the amplifier to the portable operations device is performed prior to disconnection of the amplifier from the host computer.

56. The method as claimed in claim 53, wherein transferring transmission of patient electroencephalographic signals from the amplifier to the host computer is performed responsive to reconnection of the amplifier to the host computer.

57. The method as claimed in claim 53, wherein transferring transmission of patient electroencephalographic signals from the amplifier to the host computer is performed after reconnection of the amplifier to the host computer.

58. The method as claimed in claim 53, further comprising transmitting data from the portable memory device to the host computer simultaneously with transmitting electroencephalographic signals from the amplifier to the host computer.

59. The method as claimed in claim 53, wherein disconnecting the amplifier includes disconnecting a cable running between the amplifier and the host computer.

60. The method as claimed in claim 53, wherein disconnecting the amplifier includes losing wireless communication between the amplifier and the host computer.

61. The method as claimed in claim 53, wherein disconnecting the amplifier includes losing wireless communication between the portable operations device and the host computer.

62. The method as claimed in claim 53, further comprising storing patient electroencephalographic signals to memory in the portable operations device after transferring transmission of patient electroencephalographic signals from the amplifier to the portable operations device.

63. The method as claimed in claim 62, further comprising removing the memory from the portable operations device after storing patient electroencephalographic signals thereto.

64. The method as claimed in claim 63, wherein the memory is a peripheral card releasably connected to and within the portable operations device.

65. The method as claimed in claim 62, further comprising transmitting patient electroencephalographic signals in the memory to the host computer after reconnecting the amplifier to the host computer.

66. The method as claimed in claim 65, wherein transmitting patient electroencephalographic signals in the memory to the host computer includes transmitting patient electroencephalographic signals to the amplifier and then to the host computer.

67. The method as claimed in claim 53, further comprising:
transmitting patient electroencephalographic signals from a patient's head to an input of the portable operations device after transferring transmission of patient electroencephalographic signals from the amplifier to the portable operations device via at least one electrode coupled to the patient and a jackbox coupled to the at least one electrode and to an input of the amplifier, wherein the at least one electrode is one type of patient physiological sensor; and
coupling at least one other type of patient physiological sensor to at least one additional input of the portable operations device.

68. The method as claimed in claim 67, further comprising transmitting additional patient physiological signals from the at least one other type of patient physiological sensor to the portable operations device.

69. The method as claimed in claim 53, further comprising transmitting video signals to the host computer.

70. The method as claimed in claim 53, further comprising transmitting audio signals to the host computer.

71. The method as claimed in claim 53, further comprising transmitting a stimulation signal to the patient.

72. The method as claimed in claim 53, wherein coupling the portable operations device to the amplifier includes mounting the amplifier upon the portable operations device.

73. A modular and portable electroencephalographic monitoring apparatus for use in conjunction with a host computer, the apparatus comprising:
a jackbox; and
an amplifier coupled to the jackbox, the amplifier having
an input communications port operable to receive electroencephalographic signals from the patient;
an expansion communications port operable to receive additional electroencephalographic signals from the patient, the amplifier releasably connectable to at least one additional amplifier and capable of receiving additional electroencephalographic signals from the at least one additional amplifier via the expansion communications port; and
an output communications port operable to transmit electroencephalographic signals from the amplifier and the at least one additional amplifier to the host computer.

74. The modular and portable electroencephalographic monitoring apparatus as claimed in claim 73, wherein the amplifier and the at least one additional amplifier are coupled together along a peripheral area network bus.

75. The modular and portable electroencephalographic monitoring apparatus as claimed in claim 73, wherein the amplifier and the at least one additional amplifier are coupled together in a daisy chain format.

76. The modular and portable electroencephalographic monitoring apparatus as claimed in claim 75, wherein the amplifier and the at least one additional amplifier are coupled together along a peripheral area network bus.

77. A method of monitoring electroencephalographic signals of a patient, comprising:
transmitting a first plurality of electroencephalographic signals from the patient to a first amplifier;
amplifying the first plurality of electroencephalographic signals by the first amplifier;
transmitting the first plurality of electroencephalographic signals to an output of the first amplifier;
coupling at least one other amplifier to an input of the first amplifier;
transmitting additional electroencephalographic signals from the patient to the at least one other amplifier;
amplifying the additional electroencephalographic signals by the at least one other amplifier;
transmitting the additional electroencephalographic signals to the input of the first amplifier via respective outputs of the at least one other amplifier; and
transmitting the additional electroencephalographic signals to the output of the second amplifier.

78. The method as claimed in claim 77, further comprising coupling the amplifier and the at least one other amplifier together along a peripheral area network bus.

79. The method as claimed in claim 77, wherein coupling the at least one other amplifier includes coupling the at least one other amplifier to the first amplifier in a daisy chain format.

80. The method as claimed in claim 79, wherein the first amplifier and the at least one other amplifier are coupled together along a peripheral area network bus.

81. A portable and wearable apparatus for monitoring electroencephalographic signals of a patient, comprising:
at least one electrode operable to detect electroencephalographic signals of the patient;
an amplifier wearable upon the patient, coupled to the at least one electrode, and adapted to receive electroencephalographic signals detected by the at least one electrode;
a housing wearable upon a patient;
a bay in the housing, in electrical communication with the amplifier to receive amplified electroencephalographic signals therefrom, and adapted to removably receive at least one peripheral card; and
at least one peripheral card removably received in the bay and to which amplified electroencephalographic signals can be transmitted from the bay.

82. The portable and wearable apparatus as claimed in claim 81, wherein the bay in the housing is in electrical communication with the amplifier via a peripheral area network bus.

83. The portable and wearable apparatus as claimed in claim 81, wherein the peripheral card is a memory card.

84. The portable and wearable apparatus as claimed in claim 81, wherein the peripheral card is a wireless transmitter card.

85. The portable and wearable apparatus as claimed in claim 81, further comprising a battery capable of powering the apparatus, whereby the apparatus and the patient wearing the apparatus can be completely untethered from other equipment.

86. The portable and wearable apparatus as claimed in claim 81, wherein the amplifier is a first amplifier, the apparatus further comprising at least one additional amplifier releasably coupled to the first amplifier and wearable upon the patient, wherein the bay is in electrical communication with the at least one additional amplifier and receives amplified electroencephalographic signals from the at least one additional amplifier via the first amplifier.

87. A method of monitoring electroencephalographic signals of a patient, comprising:
transmitting electroencephalographic signals from the patient to an amplifier worn on the patient;
amplifying the electroencephalographic signals by the amplifier on the patient;
transmitting the electroencephalographic signals from the amplifier to a communications bay worn on the patient;
transmitting the electroencephalographic signals from the communications bay on the patient to a removable peripheral card in the bay;
removing the peripheral card from the bay following transmission of the electroencephalographic signals to the peripheral card; and
installing another peripheral card in the bay.

88. The method as claimed in claim 87, wherein the communications bay is located in a housing worn by the patient.

89. The method as claimed in claim 88, wherein electroencephalographic signals are transmitted to the bay via a controller.

90. The method as claimed in claim 89, wherein the controller is a microprocessor.

91. The method as claimed in claim 89, further comprising:
transmitting light sensor signals from a light sensor to the communications bay; and
transmitting the light sensor signals from the communications bay to the peripheral card in the bay.

92. The method as claimed in claim 89, further comprising:
transmitting sound signals from a microphone to the communications bay; and
transmitting the sound signals from the communications bay to the peripheral card in the bay.

93. The method as claimed in claim 89, wherein the electroencephalographic signals are one type of patient physiological signal, the method further comprising:
transmitting at least one other type of patient physiological signal from the patient to the communications bay; and
transmitting the at least one other type of patient physiological signal from the communications bay to the peripheral card in the bay.

94. The method as claimed in claim 87, further comprising transmitting video signals to the host computer.

95. The method claimed in claim 87, further comprising transmitting audio signals to the host computer.

96. The method as claimed in claim 87, further comprising transmitting stimulation signals to the patient.

97. The method as claimed in claim 87, wherein the peripheral card is a memory card, the method further comprising saving the electroencephalographic signals upon the memory card.

98. The method as claimed in claim 87, wherein the peripheral card includes a wireless transmitter, the method further comprising transmitting electroencephalographic signals from the peripheral card.

99. The method as claimed in claim 87, wherein electroencephalographic signals are transmitted from the amplifier to the communications bay via a peripheral area network bus.

100. The method as claimed in claim 87, wherein the amplifier is a first amplifier, the method further comprising coupling at least one additional amplifier to the first amplifier.

101. The method as claimed in claim 100, wherein the at least one additional amplifier is coupled to the first amplifier in a daisy chain format.

102. The method as claimed in claim 100, further comprising:
transmitting additional electroencephalographic signals from the at least one additional amplifier to the first amplifier; and
transmitting the additional electroencephalographic signals from the first amplifier to the communications bay.

103. An electroencephalographic signal display apparatus, comprising:
a handheld portable housing;
an input operable to receive electroencephalographic signals of a patient;
a display coupled to the input and operable to display data representative of the electroencephalographic signals;
a controller in the housing and coupled to the input and display, the controller adapted to selectively display at least one of the electroencephalographic signals received from the input; and
at least one user-manipulatable control on the housing, coupled to the controller, and operable to change the electroencephalographic signals displayed by the controller upon the display.

104. The electroencephalographic signal display apparatus as claimed in claim 103 for use in conjunction with an electroencephalographic monitoring device having at least one electrode, the apparatus further comprising an output operable to transmit control signals to the electroencephalographic monitoring device to control operation thereof.

105. The electroencephalographic signal display apparatus as claimed in claim 104, wherein the input and output are coupled to the electroencephalographic monitoring device via a cable.

106. The electroencephalographic signal display apparatus as claimed in claim 103 for use in conjunction with an electroencephalographic monitoring device coupled to the apparatus, wherein the apparatus is adapted to be releasably connectable to the electroencephalographic monitoring device via a peripheral area network bus.

107. The electroencephalographic signal display apparatus as claimed in claim 103 for use in conjunction with at least one electrode coupled to the apparatus, the apparatus further comprising an operational mode in which electroencephalographic signals from the at least one electrode are displayed upon the display.

108. The electroencephalographic signal display apparatus as claimed in claim 103, further comprising a controller operable to display at least one trace of a patient's electroencephalographic signals upon the display.

109. The electroencephalographic signal display apparatus as claimed in claim 103 for use in conjunction with at least one electrode coupled to the apparatus, the apparatus further comprising an operational mode in which impedance of at least one electrode is displayed upon the display.

110. The electroencephalographic signal display apparatus as claimed in claim 103 for use in conjunction with at least one electrode coupled to the apparatus, wherein the controller is operable by the at least one user-manipulatable control to change a threshold impedance of the at least one electrode.

111. The electroencephalographic signal display apparatus as claimed in claim 103 for use in conjunction with a pulse oximeter coupled to the apparatus, the apparatus further comprising a pulse oximeter mode in which pulse oximeter signals from the pulse oximeter are displayed upon the display.

112. The electroencephalographic signal display apparatus as claimed in claim 103, for use in conjunction with multiple amplifiers, wherein the input is operable to receive electroencephalographic signals of the patient from multiple amplifiers coupled together in a daisy chain format.

113. The electroencephalographic signal display apparatus as claimed in claim 112, wherein the apparatus is adapted to be releasably coupled to at least one amplifier via a peripheral area network bus.

114. A method of displaying electroencephalographic signals, comprising:
transmitting electroencephalographic signals from a patient to an input of a handheld display device;
transmitting the electroencephalographic signals from the input to a controller in the handheld display device;
displaying electroencephalographic signal information regarding the electroencephalographic signals upon a display on the handheld display device via the controller;
receiving at least one display command from a user;
transmitting the at least one display command to the controller; and
changing the electroencephalographic signal information being displayed upon the display in response to transmitting the at least one display command to the controller.

115. The method as claimed in claim 114, further comprising wearing the handheld display device upon the patient.

116. The method as claimed in claim 115, further comprising coupling the handheld display device to at least one amplifier worn upon the patient and from which the electroencephalographic signals are received.

117. The method as claimed in claim 114, wherein transmitting electroencephalographic signals from the patient to the input of the handheld display device occurs over a peripheral area network bus.

118. The method as claimed in claim 114, wherein displaying electroencephalographic signal information includes displaying the threshold impedance value of at least one electrode on the patient.

119. The method as claimed in claim 118, further comprising:
receiving commands from a user via at least one user-manipulatable control on the handheld display device; and
displaying threshold impedance values of different electrodes on the patient in response to receiving commands from the user.

120. The method as claimed in claim 118, further comprising:
receiving commands from a user via at least one user-manipulatable control on the handheld display device; and
changing desired threshold impedance values of electrodes on the patient in response to receiving commands from the user.

121. The method as claimed in claim 114, wherein displaying electroencephalographic signal information includes a display mode in which electroencephalographic information is displayed only for electrodes upon the patient having threshold impedance values that have been exceeded.

122. The method as claimed in claim 121, further comprising:
transmitting updated electroencephalographic signals to the input of the handheld display device;
transmitting the updated electroencephalographic signals from the input to the controller; and
continuously updating the display mode with updated electroencephalographic signal information regarding the updated electroencephalographic signals received by the input and controller.

123. The method as claimed in claim 114, further comprising:
transmitting updated electroencephalographic signals to the input of the handheld display device;
transmitting the updated electroencephalographic signals from the input to the controller; and
continuously updating the display with updated electroencephalographic signal information regarding the updated electroencephalographic signals received by the input and controller.

124. The method as claimed in claim 114, wherein displaying electroencephalographic signal information includes displaying at least one trace of a patient's electroencephalographic signals corresponding to at least one respective electrode coupled to the handheld display device.

125. The method as claimed in claim 114, further comprising:
transmitting pulse oximetry signals from a pulse oximeter coupled to the patient to the input of the handheld display device;
transmitting the pulse oximetry signals from the input to the controller in the handheld display device; and
displaying pulse oximetry signal information regarding the pulse oximetry signals upon the display on the handheld device via the controller.

126. A wearable electroencephalographic monitoring apparatus for use in conjunction with a host computer, comprising:
at least one electrode operable to receive electroencephalographic signals from a patient;
a jackbox coupled to at least one electrode;
an amplifier releasably coupled to the jackbox and to the host computer;
a communications device having
a housing;
an input coupled to the amplifier;
an output coupled to the host computer; and
a controller operable to selectively route electroencephalographic signals received from the amplifier to the output of the communications device; and
a battery coupled to the communications device;
wherein the amplifier, communications device, and battery are arranged as a single physically integrated unit wearable by the patient.

127. The wearable electroencephalographic monitoring apparatus as claimed in claim 126, wherein the input and output are defined by a communications port between the communications device and the amplifier.

128. The wearable electroencephalographic monitoring apparatus as claimed in claim 126, wherein:
the jackbox is releasably mounted upon the amplifier; and
the jackbox, amplifier, communications device, and battery are arranged as a single physically integrated unit wearable by the patient.

129. The wearable electroencephalographic monitoring apparatus as claimed in claim 126, further comprising a holster within which the single physically integrated unit is at least partially received.

130. The wearable electroencephalographic monitoring apparatus as claimed in claim 129, further comprising a patient-wearable belt upon which the holster is coupled.

131. The wearable electroencephalographic monitoring apparatus as claimed in claim 126, further comprising a belt upon which the single physically integrated unit is coupled.

132. The wearable electroencephalographic monitoring apparatus as claimed in claim 126, wherein the jackbox and the amplifier have a common housing.

133. The wearable electroencephalographic monitoring apparatus as claimed in claim 126, wherein the communications device further includes a peripheral card to which the controller can selectively route electroencephalographic signals.

134. The wearable electroencephalographic monitoring apparatus as claimed in claim 133, wherein the peripheral card is removable from the communications device.

135. The wearable electroencephalographic monitoring apparatus as claimed in claim 126, wherein the apparatus is releasably tethered to and in communication with the host computer via a single cable coupled to the amplifier.

136. The wearable electroencephalographic monitoring apparatus as claimed in claim 126, wherein the amplifier is a first amplifier, the apparatus further comprising at least one additional amplifier coupled to the first amplifier to transmit electroencephalographic signals from each at least one additional amplifier to the host computer via the first amplifier.

137. The wearable electroencephalographic monitoring apparatus as claimed in claim 136, further comprising:
a belt wearable by the patient and to which the amplifier and the at least one additional amplifier are coupled; and
at least one cable coupled to the belt and connecting the first amplifier and the at least one additional amplifier together.

138. A method of monitoring electroencephalographic signals of a patient, comprising:
providing an amplifier operable to receive electroencephalographic signals from the patient;
mounting the amplifier upon a housing within which is located a controller;
coupling a battery to the housing;
wearing the amplifier, housing, and battery upon the patient as a single physically integrated unit;
transmitting electroencephalographic signals from the patient to the amplifier;
transmitting electroencephalographic signals from the amplifier to the controller; and
transmitting electroencephalographic signals from the controller to a host computer.

139. The method as claimed in claim 138, wherein transmitting electroencephalographic signals from the controller to the host computer includes transmitting electroencephalographic signals from the controller to the amplifier and then to the host computer.

140. The method as claimed in claim 138, further comprising:
coupling at least one electrode to the patient and to a jackbox; and
mounting the jackbox upon the amplifier.

141. The method as claimed in claim 138, wherein mounting the amplifier upon the housing establishes electrical communication between the amplifier and the controller.

142. The method as claimed in claim 138, wherein coupling the battery to the housing includes mounting the battery upon the housing.

143. The method as claimed in claim 142, further comprising:
coupling at least one electrode to the patient and to a jackbox; and
mounting the jackbox upon the amplifier.

144. The method as claimed in claim 138, further comprising coupling at least one electrode to the patient and to a jackbox, wherein the jackbox and the amplifier have a common housing.

145. The method as claimed in claim 138, wherein wearing the amplifier, housing, and battery upon the patient as a single physically integrated unit includes inserting at least part of the unit in a holster worn upon the patient.

146. The method as claimed in claim 145, wherein wearing the amplifier, housing, and battery upon the patient as a single physically integrated unit also includes coupling the holster to a belt worn by the patient.

147. The method as claimed in claim 138, wherein wearing the amplifier, housing, and battery upon the patient as a single physically integrated unit includes coupling the unit to a belt worn by the patient.

148. The method as claimed in claim 138, further comprising coupling the unit to the host computer by a single cable coupled to the amplifier prior to transmitting electroencephalographic signals from the controller to a host computer

149. The method as claimed in claim 138, further comprising coupling at least one removable peripheral card to the controller.

150. The method as claimed in claim 138, wherein the amplifier is a first amplifier, the method further comprising:
coupling at least one additional amplifier to the first amplifier;
transmitting additional electroencephalographic signals from the patient to the at least one additional amplifier;
transmitting the additional electroencephalographic signals from the at least one additional amplifier to the first amplifier;
transmitting the additional electroencephalographic signals from the first amplifier to the controller; and
transmitting the additional electroencephalographic signals from the controller to the host computer.

151. The method as claimed in claim 150, further comprising:
coupling the first amplifier and the at least one additional amplifier to a belt worn by the patient;
coupling the first amplifier and the at least one additional amplifier together by at least one cable; and
coupling the at least one cable to the belt.
